(19) United States
(12) Patent Application Publication
(10) Pub. No.: US 2023/0061229 A1
(43) Pub. Date: Mar. 2, 2023

(54) SMART WOUND DRESSINGS

(71) Applicants: INTELIGELS LTD., Hoshaya (IL); YISSUM RESEARCH DEVELOPMENT COMPANY OF THE HEBREW UNIVERSITY OF JERUSALEM LTD., Jerusalem (IL)

(72) Inventor: Daniel COHN, Jerusalem (IL)

(21) Appl. No.: 17/758,318

(22) PCT Filed: Jan. 2, 2021
(86) PCT No.: PCT/IL2021/050008

§ 371 Date: Jul. 1, 2022

Related U.S. Application Data
(60) Provisional application No. 62/956,390, filed on Jan. 2, 2020.
(60) Provisional application No. 63/083,936, filed on Sep. 27, 2020.

(51) Int. Cl. A61L 26/00

(52) U.S. Cl. CPC A61L 26/008; A61L 26/0066; A61L 26/0019; A61L 2300/604

(57) ABSTRACT

The present invention discloses either or both in situ and a priori generated hydrogel wound dressings comprise one or more RTR components in low viscosity aqueous solution and one or more non-RTR components. A dressing, comprising at least one first RTR and an active component (AC) integrated within the RTR is also disclosed. The invention further discloses a method of treating a medical or cosmetic indication by a wound dressing, comprising either or both in situ and a priori generating hydrogel wound dressings by providing one or more RTR components in low viscosity aqueous solution and one or more non-RTR components. it and methods for treating a medical or cosmetic indication by providing a dressing with at least one first RTR and with at least one active component (AC) integrated within the RTR are also disclosed.

### FIELD AND BACKGROUND OF THE INVENTION

The present invention generally pertains smart wound dressings and to methods thereof.

There is a wide variety of materials which are foreign to the human body and which are used in direct contact with its organs, tissues and fluids. These materials are called Biomaterials, and among them, polymers play a pivotal role. An important category of biomaterials constitutes the basis of biomedical systems that come in intimate contact with tissues and that need to be removed in due time. This class of systems includes, among others, wound and burn dressings, temporary sealants, bandages and gauzes.

The number of wound care products has increased dramatically over the last two decades, and they play a key role in the treatment of the many different classes of wounds known. During this period also new strategies were introduced, most importantly Tissue Engineering based dressings, which perform also as the scaffold for the regeneration of autologous tissues.

The term “wound dressing” is used throughout this document as covering not only wound dressings in the form of classical bandages, but as a generic term covering also burn dressings of all types, as well as bandages, gauzes, non-woven fabrics, hemostats, temporary sealants, and any other biomedical system that is used in direct contact with tissue or organs, pre-formed or in situ generated, and that requires to be detached from it in a non-injurious and gentle way in due course, in some cases, repeatedly and frequently.

One of the key problems shared by existing wound dressings is the detrimental effect that removing the dressing has on the healing of the wound and/or the integrity of the tissue.

The removal of the dressing after a period of intimate contact with the wounded tissue and its secretions results in the tight adherence of the dressing to the tissue. The removal of the dressing or each dressing replacement is, therefore, not only very painful but also, and even more importantly, results in tearing and hurting the wound bed, significantly erasing the progress made since the previous dressing change.

For the sake of clarity, conciseness and simplicity, and without detracting from the generality of the scope of the present invention in any form or fashion, the inventors have chosen to illustrate the invention hereby disclosed, by focusing on its biomedical applications, and more specifically, to illustrate their use as a wound dressing (as defined above) to be used in the human or animal body. This, even though the polymeric compositions disclosed hereby can be applied in a diversity of non-biomedical applications, where a temporary interface between a substrate and another material is initially generated, and then, triggered by environmental stimuli and or in due time and “on command”, the substrate and the material need to be easily separated, by affecting the interfacing layer. In the case selected by the inventors to illustrate the invention, namely, the biomedical field, the working temperature (T_{w}) of these compositions is the relevant body temperature (T_{b}) at the site of performance of the dressing. For the sake of clarity, conciseness and simplicity, and without detracting from the generality of the scope of the present invention in any form or fashion, the term wound dressing will be used throughout this document, to indicate all the systems, without limitation, covered by the invention disclosed hereby.

The present invention discloses a new type of wound dressing displaying advantageous features, which comprises at least one environmentally responsive component, the environmentally responsive component being capable of reversion between two sates: an attachable state at the physiological conditions at its site of performance, when in contact with a tissue or an organ, and a detachable interface for easy removal from the tissue or organ upon application of the environmental stimuli.

The environmental stimulus may be temperature, pH or other stimuli such as ionic strength or light of various wavelengths, among others, and combinations thereof.

For the sake of clarity, conciseness and simplicity, and without detracting from the generality of the scope of the present invention in any form or fashion, the inventors have chosen to illustrate the invention hereby disclosed, by focusing on its use as a wound dressing, where the environmental stimulus is temperature. The description of the invention, as presented below will, therefore, and without detracting from the generality of the scope of the present invention in any form or fashion, confine itself to wound dressings that comprise at least one thermo-responsive component and, more specifically reverse thermo-responsive (RTR) materials, preferable polymers.

Contrary to the prior art, the invention disclosed hereby produces novel wound dressings that consist, partially or totally, of RTR materials, preferably polymers, that will impart advantageous properties to the final wound dressing. By selecting and controlling the chemical and mechanical properties of both, the RTR component/s and the non-RTR component/s, (should the dressing comprise also non-RTR component/s,) as well as the ratio between the two types of constituents, namely RTR and non-RTR, the different properties of the wound dressing can be fine-tuned.

Hydrogels are considered today one of the leading types of wound dressings, since they combine the advantageous attributes of moist wound healing. Among them is worth mentioning their enhanced biocompatibility, the minimal inflammatory response and thrombosis they elicit, and the marginal tissue damage they cause. Additionally, hydrogels are characterized by high permeability levels of oxygen, nutrients and other water-soluble metabolites, and they also display tunable fluid absorbance levels and superior transparency.

Even though not of universal applicability, in situ generated hydrogels have key advantages when compared to pre-formed ones, most importantly due to their high conformability to the shape of the wound, regardless of its complexity, avoiding also wrinkling or folding of the dressing. Their ease of deployment, their universal adaptability and the comfort they offer the patient are additional advantageous features of in situ formed hydrogels. Moreover, since the administration of in situ formed hydrogel dressings is rapid, clean, hands free and straightforward, higher patient levels of compliance are achieved.

Hydron is a dressing based on polyhydroxyethyl methacrylate (PHEMA) microparticles and liquid polyethylene glycol (PEG) 400 that is formed in situ by spraying the gel on the wound. This early gel was flawed with important drawbacks, most importantly its cumbersome application and the fact that the water soluble PEG400 is absorbed by the wound over time, leaving behind an increasingly stiff and brittle PHEMA covering. These shortcomings substantially limited its use.

A dressing consisting of a blend of hyaluronic acid and chrondroitin sulphate derivatized with adipic dihydrazide was formed on the wound by crosslinking it with polyethylene glycol propiodialdehyde. A gelatin-based spray-on foam bandage is yet another example of attempts to develop in situ formed dressings. In another study, Balakrishnan et al demonstrated that small concentrations of borax speed up the reaction between periodate-oxidized alginate and gelatin, to produce the hydrogel on the wound. In another contribution of the same group, an oxidized alginate-gelatin blend was proposed as the basis of an in situ wound dressing, which was reported to display some of the properties required.

In light of their theoretical advantageous features, as enumerated above, the small number of in situ generated wound dressings currently in the clinic, is surprising. The most important problems of the in situ generated wound dressings investigated to date, including those currently in the clinic pertain to the fact that their vast majority convert the liquid into a gel via polymerization or crosslinking reactions. The drawbacks of this approach stem from the fact that the reactions used are typically unacceptably slow for this application. Even more importantly, these reactions entail the use of hazardous toxic compounds. To exemplify this serious problem, suffice to mention the in situ formed dressing prepared by reacting gelatin and oxidized alginate in the presence of a small amount of borax. In this case, the crosslinking reaction forms toxic alginate di-aldehyde, similarly to the toxicity encountered when crosslinking bioprostheses with glutaraldehyde. Additional drawbacks of these dressings stem from the fact that in most cases they are not user friendly and cumbersome to form, since most of them require mixing two components to administer it. Furthermore, having to mix two components may make the dressing also not homogeneous and not reproducible.

An alternative strategy proposed to avoid this important problem capitalizes on the unique behavior of environmentally responsive polymers, especially RTR ones. Early work conducted at the beginning of the 1970s explored the possibility of using polyethylene oxide-polypropylene oxide-polyethylene oxide (PEO-PPO-PEO) triblocks able to gel at physiological temperature, to treat burns in rats. Due to crucial limitations of the materials used, one of them being their extremely long time of gelation, these early studies did not result in any further development. Recently, Lina Du and coworkers tested a combination of the same type of PEO-PPO-PEO triblocks, seeking only to shorten the gelation time. These in situ generated gels displayed very poor properties, including their very low viscosity, short residence times and unacceptable weakness. It is also worth stressing that in none of these studies of the prior art, removal of the dressing by cooling and liquefication was considered, this being a unique, key feature of the dressing disclosed by this invention.

Spray-on films were the first in situ generated coverings, introduced already back in the 1950s, but they all generate hydrophobic polymeric films, being flawed with all the drawbacks of essentially hermetic dressings. The fact that they can be used only on abrasions and small cuts, further limits their clinical impact. Moreover, their removal is difficult, time-consuming and most of them require an organic solvent to be removed. The spray-on films presently in clinical use are marred with important additional shortcomings and limitations—some of them dangerous—that pertain to all three stages of their use: deployment, performance and removal. The most far-reaching drawback is that the spray-on products are applied out of a polymeric solution comprising a highly volatile organic solvent. Due to the presence of the organic solvent, its volatility, flammability and toxicity, the manufacturer's “Instructions for Use” explicitly state that much caution should be exercised when using spray-on films. Among other cautionary statements, the manufacturers require the user to avoid inhaling while applying the dressing, to deploy it only in well ventilated areas and avoid contact with the eyes. Furthermore, the manufacturers also instruct the user to apply the dressing far from of a naked flame and any incandescent material. Because of the volatile and flammable solvent, also smoking should be avoided when administering the product. Suffice to add that these products should also be kept out of reach of children, to fully appreciate the drawbacks and limitations of the spray-on films currently in the market. Due to the importance of its large negative effect on the wound, as already stressed, spray-on products form hydrophobic polymeric films on the wound, having, therefore, all the shortcomings of non-hydrogel dressings.

All dressings, regardless of their type, share a common, ever-present fact: unavoidably, all of them have to be removed in due time and replaced by a new one. The removal of the dressing after a period of intimate contact with the wounded tissue and its secretions, results in the tight adherence of the dressing to the tissue bed. Each dressing change is, therefore, not only extremely painful but also, and most importantly, results in tearing and injuring the wound, significantly erasing the progress made by the healing process, since the previous dressing change.

The size of the wound dressing market has expanded substantially during the last decade, mainly due to [i] an increase of the elder segment of the population and [ii] a steady rise in the incidence of diseases, mainly obesity and diabetes. A recent survey determined that the 2016 global wound dressings market was around 6.3 billion dollars, while the forecast anticipates a steady expansion, with the market being expected to surpass 8.5 billion dollars in 2021.

“Smart” polymers are an advanced class of materials tailored to display substantial property changes, as a response to minor chemical, physical or biological stimuli, such as temperature, pH, biochemical agents, mechanical stresses, electrical or magnetic fields, ionic strength and irradiation.

The term “thermo-sensitive” refers to the ability of a polymeric system to achieve significant chemical, mechanical, physical or biological changes and combinations thereof, due to small temperature differentials. The resulting change is based on different mechanisms such as ionization and entropy gain due to water molecules release, among others (Alexandridis and Hatton, Colloids and Surfaces A, 96, 1 (1995)).

Thermo-sensitive gels can be classified into two categories: (a) If they have an upper critical solution temperature (UCST), they are named positive-sensitive hydrogels and they contract upon cooling below the UCST, or (b) If they have a lower critical solution temperature (LCST), they are called negative-sensitive hydrogels and they contract upon heating above this temperature.

The reverse thermo-responsive (RTR) phenomenon constitutes one of the most promising strategies for the development of easily deployable systems, such as, without limitation, injectables. The water solutions of these materials display low viscosity at a low temperature, below or at ambient temperature, and exhibit a sharp viscosity increase as temperature rises within a very narrow temperature interval, producing a semi-solid gel once they reach body temperature. This phenomenon is harnessed in the invention disclosed hereby, to produce in situ generated dressings that are deployed as solutions and sprays, among other modalities of deployment that will gel once in contact with tissues, generating topically or inside the body, a dressing. The dressing will also be easily removed by cooling and liquefying it.

There are several RTR displaying polymers. The compositions of the present invention include combinations of any type of reverse thermo-responsive materials such as poly(ethylene oxide)-poly(propylene oxide)-poly(ethylene oxide) (PEO-PPO-PEO) triblocks, random or alternating reverse thermo-responsive PEO-PPO block copolymers such as those described e.g., by Cohn, Sosnik and Kheyfetz in U.S. Patent Published number US 2003/(the teachings of which are incorporated herein by reference), N-alkyl substituted acrylamides (preferably poly-N-isopropyl acrylamide [PNIPAAm], cellulose derivatives, selected from a group consisting of hydroxypropyl methylcellulose and hydroxypropyl cellulose, alternating or random, and various amphiphilic polymers such as poly(ethylene oxide)-polylactic acid block copolymers. Poly(N-isopropyl acrylamide) (PNIPAAm) is one of the most widely used thermo-responsive polymers [Tanaka and co-workers in U.S. Pat. No. 5,403,893 and Hoffman A. S. et al., J. Controlled Release, 6, 297 (1987)].

The family of poly(ethylene oxide)/poly(propylene oxide)/poly(ethylene oxide) (PEOPPO-PEO) triblocks, commercially available as Pluronic®™ (Krezanoski in U.S. Pat. No. 4,188,373) is another important example of RTR-displaying polymers. The {EO}₉₉-{PO}₆₇-{EO}₉₉ triblock, known as Pluronic F127, has attracted much attention due to the properties of the gels it generates Adjusting the concentration of the polymer, renders the solution with the desired liquid-gel transition. An additional system which is liquid at room temperature, and becomes a semi-solid gel when warmed to about body temperature, is described in U.S. Pat. No. 5,252,318, and consists of tetrafunctional block polymers of polyoxyethylene and polyoxypropylene condensed with ethylenediamine (commercially available as commercially available Tetronic®™). The endothermic phase transition taking place is driven by the entropy gain caused by the release of water molecules bound to the hydrophobic groups in the polymer backbone.

Polymeric surfaces can be rendered “smart” (or tunable or switchable or environmentally-responsive), being able to respond in various ways to minor changes in their environment. Among others, their responsiveness to changes in temperature, pH, light and solvent characteristics, have attracted much attention. The environmentally triggered spatial reorganization of these surfaces can by utilized to tailor various properties pertaining to their use in areas such as drug delivery, biomimetic materials, tribology and chemical gates.

The temperature-activated reversible coiling and uncoiling of water-soluble polymeric chains attached to the surface of a substrate represents one of the most promising strategies to engineer switchable surfaces. Surfaces grafted with PNiPAAm chains, for example, exhibit a remarkable change in their hydrophilic/hydrophobic nature, due to the remarkable interfacial mobility of PNiPAAm hydrated molecules. As expected, given the restricted mobility of the covalently bound chains, their hydration/dehydration transition occurs at a lower temperature, when compared to PNiPAAm free molecules. The same is applicable to other RTR chains such as those comprising polyoxyethylene and polyoxypropylene segments.

### SUMMARY OF THE INVENTION

The fundamental feature of the invention disclosed hereby is the creation of a tissue or organ contacting, stimulus-responsive interface, that makes the deployment and removal of the dressing safe, efficacious, devoid of pain and, most importantly, minimally disruptive to the healing and repair processes. The generation of an in situ generated conformable interface between the wound dressing and the tissue is another embodiment of the invention disclosed hereby.

The RTR component/s may be connected or not to the other components of the wound dressing of the invention. The RTR component/s of the invention may also interact with other materials, devices, implants, prostheses and tissues, among others, present temporarily or permanently at the site of treatment.

The different components of the invention disclosed hereby may be natural or synthetic.

In addition to the pre-formed dressing taught by this invention, in situ generated, easily deployable hydrogel dressings, displaying the required mechanical, adhesive, transport and optical properties, that can then be simply liquefied by cooling it and be removed without injuring the tissue and hampering the healing process, and without causing pain to the patient, are key embodiments of the invention disclosed hereby.

These novel in situ generated hydrogel wound dressings, named Smart Liquefiable Dressings (SLD) comprise environmentally responsive polymers, preferably RTR polymers.

Another embodiment the invention concerns a composition to be applied to a desired site (such as, without limitation, on a wound or burn site, among others) that serves as an interface between the wound and a subsequently applied dressing, of any type. According to this embodiment, the composition may be preferably a topical composition or used internally. In accordance with this embodiment the RTR component/s is applied on the wound as a low viscosity aqueous solution. In embodiments of the present invention, the RTR component may be applied prior to, simultaneously or after other non-RTR components.

An in situ generated dressing consisting partially or entirely of RTR displaying materials that gels and generates a dressing upon deployment, as it becomes in contact with the tissue, is yet another preferred embodiment of this invention. The in situ generated RTR displaying dressing may comprise also additional materials, having different configurations, for different purposes such as, without limitation, to optimize its viscosity, to enhance the dressing attachment to the different tissues it comes in contact with, and to enhance the ability of the dressing to retain its water content.

The dressings, especially the in situ generated ones disclosed hereby, are expected to suitably attach to the wound bed due to their low viscosity during deployment that will enable them to penetrate the pores and crevices of the wounded tissue and fully conform to it. That said, seeking to maximize their stability on the tissue bed, the dressings may also be rendered mucoadhesive by adding to the RTR aqueous solution a mucoadhesive or tissue adhesive component, water soluble or not, such as polyacrylic acid or chitosan, among others.

The RTR polymers used in this invention are thermoplastics or thermosets, or combinations thereof, provided the key requirement of “on command” liquefiability is retained. In some embodiments, the RTR system combines the straightforward liquefiability of thermoplastic RTR polymers and the superior mechanical properties of their thermoset counterparts.

The RTR component/s or any other component part of the invention disclosed hereby, should the composition comprise more than one RTR component and a non-RTR component, may be applied in one shot or sequentially. The various components of the invention taught hereby, RTR or non-RTR, may have the same or a different composition and/or concentration and/or molecular weight. The compositions disclosed hereby may also comprise components that are responsive to different environmental stimuli or may perform other tasks, such as, without limitation, other chemical, physical, mechanical or biological functions and combinations thereof. The composition and properties of the RTR and/or non-RTR component/s may also vary along any of the axes of the dressing, being different at different points of the wound.

Any of the components, RTR or not, may be administered simultaneously or after any of the other components of the invention.

In another embodiment, the RTR component/s may be connected between them and/or connected to non-RTR component/s so as to form an integral part of the wound dressing. The connection between the two types of components may be covalent, ionic, physical entrapment or any other type of binding. In a preferred embodiment, the RTR component/s of the invention disclosed hereby may form, without limitation, an RTR layer. In a preferred embodiment, the layer may consist, without limitation, partially or exclusively, of RTR-displaying chains attached, covalently or otherwise, to the surface of a suitable substrate. Typically, the RTR layer will be deployed at a temperature below the relevant thermal transition and will gel, partially or totally, sharply or gradually, as the system heats up to the temperature of the site. Towards removal, the dressing is cooled down, below the relevant thermal transition of the RTR gel, substantially weakening the interfacial layer and, typically, liquefying it (partially or fully), making the detachment of the wound dressing from the wound bed and/or of the surrounding tissues easy, not painful, as well as not injurious to the tissue.

The invention taught hereby discloses a multi-component wound dressing that can be replaced easily and rapidly by lowering the temperature and simply liquefying, partially or fully, the gel that interfaces with the wound bed that then disengages from the tissue without traumatizing the wound and without pain.

The invention disclosed hereby displays highly advantageous features relevant to all patients and all biomedical systems that come in intimate contact with tissues and organs, that need to be removed in due time. Having the that, the unique feature of the invention taught hereby is of a critical importance and special benefit for patients with specific pathologies such as, without limitation, diabetes and hemophilia, where even minor bleeding may become a major complication, or in situations where bleeding should be avoided due to the spreading of infectious or other diseases.

The RTR component/s and the non-RTR component/s may be not connected in any way, or may generate bonds between each category of constituents, namely, the RTR component/s among themselves and the non-RTR component/s among themselves or there may be also any kind of binding between some or all of the RTR component/s and some or all of the non-RTR component/s. In another embodiment taught by this invention, the wound dressing may comprise also components that respond to different stimuli, for the purpose of deploying and removing the dressing and/or for any other purpose. In another embodiment of this invention, one component may respond to one stimulus and another component to another stimulus. Any of the components of the compositions disclosed by this invention may respond to one or more stimuli whereby, for example, without limitation, one or more therapeutic agents are released.

The dressing may consist, partially or totally, of blends, semi-IPNs, IPNs or copolymers, including either all or part of the RTR component/s and all or part of the non-RTR component/s, in any combination. The dressing may be uniform and homogeneous in space or may consist of different zones having different compositions and/or different molecular weights and/or thickness and/or displaying different properties, these zones being able to be nanometric, up to macroscopic, continuous or discontinuous, creating independent or interconnected domains within the system, having several geometries, architectures and spatial arrays, dispersed homogeneously or heterogeneously, isotropically or anisotropically.

It is a preferred object of this invention to generate products that consist of more than one layer.

In another embodiment, the wound dressings disclosed hereby comprise also an additional solid component/s that can appear in a diversity of shapes, sizes and geometries, including, without limitation, spheres, particles of any other shape, capsules, fibers, ribbons, films, meshes, fabrics, non-woven structures, foams, porous structures of different types, each of them having the possibility of being solid, porous, hollow and/or combinations thereof. These solid component/s may be solid already at deployment time or they may be generated and/or solidify in situ, during or immediately after deployment or later on, over time. The solid component/s may differ significantly, without limitation, in their composition, behavior and in their different properties.

These components may also be present as solids for the whole period during which the wound dressing is at the site, or, without limitation, they may also change their composition over time and/or may degrade and/or swell and/or dissolve and/or crosslink, and combinations thereof.

In another embodiment, any of the component/s of this invention may include a component/s of pharmacological and/or biological relevance, such as, among many others, and without limitation, drugs and drug residues, oligopeptide sequences, growth factors, material containing genetic information and combinations thereof. These component/s of pharmacological and/or biological relevance may be just blended with any of the component/s of the system, prior to, during or after deployment, and/or may be attached, covalently or otherwise, to one or more of the RTR component/s and/or non-RTR component/s of the system.

It is another preferred embodiment of this invention, to comprise more than one environmentally responsive component/s, such as, among others, and without limitation, being RTR or pH-sensitive, or respond to other environmental stimuli, such as, among others, and without limitation, ionic strength, light, electrical and/or magnetic fields, and combinations thereof.

Biocompatibility and serializability are two additional important attributes of the materials disclosed hereby. Furthermore, the materials comprising this invention as well as their degradation products, should they comprise biodegradable components, are not harmfully toxic and, therefore, can be used both on the surface of the body as well as internally.

In another embodiment of the present invention, polymeric chains displaying the temperature-dependent coiling-uncoiling ability in aqueous media are attached to the surface of a substrate, typically polymeric.

In a preferred embodiment the wound dressing is applied wet and cold, when the chains present on its surface are in their expanded conformation. Once at the site, the surface layer of RTR chains will coil, generating a surface-attached gel layer. In a preferred embodiment, towards removal, the wound dressing is cooled down, below the temperature where the chains uncoil, allowing a smooth and not painful removal of the wound dressing and one that does not cause trauma to the tissue bed. The RTR chains may be covalently grafted onto the surface and/or attached in any other way, such as, and without limitation, by generating hydrogen bonds, ionic bonds, complexation or by mechanical interlocking with the substrate.

In another embodiment, this invention includes additional components so that it also promotes the regeneration of healthy tissue at the site.

It is also an object of the compositions of the present invention to include also additional materials that fulfill other roles, including, without limitation, rendering the system with the desired mechanical behavior or with the appropriate transport properties or with the suitable attachment behavior to the tissue bed, or any other desirable and advantageous chemical, physical or biological characteristics, and combinations thereof.

It is a preferred object of this invention to engineer wound dressings that consist of more than one layer. In one embodiment, a tissue facing layer is formed, aimed at a two-fold objective: maximizing its tight attachment to the site, following the deployment stage and, conversely, minimizing its attachment to the site and allowing its removal to be minimally disruptive to the tissue, when required.

In one embodiment, this is achieved, without limitation, by rendering the tissue contacting layer RTR. The RTR materials are preferably polymeric, and among them, without limitation, polymers such as poly(ethylene oxide)-poly(propylene oxide)-poly(ethylene oxide) (PEO-PPO-PEO) triblocks, random or alternating RTR PEO-PPO block copolymers, tetrafunctional block polymers of polyoxyethylene and polyoxypropylene condensed with ethylenediamine, N-alkyl substituted acrylamides (preferably poly-N-isopropyl acrylamide [PNIPAAm], cellulose derivatives, selected from a group consisting of hydroxypropyl methylcellulose and hydroxypropyl cellulose, alternating or random, and various amphiphilic polymers such as poly(ethylene oxide)-polylactic acid block copolymers, and combinations thereof.

It is another embodiment of this invention that any of the components of the compositions disclosed hereby, RTR and/or non-RTR, is able to retain and release active molecules over time. In a preferred embodiment taught by this invention, when an RTR component is loaded and releases active molecules over time, the loading may be conducted at a lower temperature, while the chains are in their expanded conformation. The release, in turn, will take place at site temperature, from the chains that are in their collapsed state. It is an additional embodiment of this invention that the temperature may be lowered as required, to allow for release “on command” of biologically active species, without removing the wound dressing. In addition to generating an easily detachable and removable dressing, it is an embodiment of this invention to generate a wound dressing that also serves as a depot and reservoir of therapeutic material to be released at a desired time and at a desired rate, in some instances by applying a suitable environmental stimulus or by any other mechanism such as diffusion with or without degradation of the relevant component.

It is another embodiment of this invention that any of the components of the compositions disclosed hereby, RTR and/or non-RTR, comprises cells that play a role in the healing and repair process. In a preferred embodiment taught by this invention, the cells may be incorporated into the RTR component/s at a lower temperature, while the system is in its low viscosity state. In another embodiment taught by this invention, the cells may be incorporated into any of the other components of the dressing. It is another embodiment of this invention that it is able to retain and release active molecules over time and also contains cells.

In another embodiment, the wound dressing disclosed hereby comprises RTR chains that have been physically entrapped in the surface of other component/s. This can be achieved, without limitation, by selectively swelling the surface layer of the other component/s with solutions of RTR polymers, followed by the removal of the solvent, typically by evaporation. The surface layer then shrinks back as the solvent is removed, entrapping the polymeric chains within the substrate surface. The advantages of this approach pertain primarily to its simplicity and its relatively straightforward incorporation into the substrate. In another embodiment, the RTR components are added to the substrate when it is amorphous state, crystallizing then and entrapping the RTR chains. In another embodiment, the RTR components are added to the substrate when it is not fully polymerized, polymerizing fully once the RTR chains are added, entrapping then the RTR chains.

It is also an object of the invention disclosed hereby to generate solid particles of the non-RTR component/s, having any geometry, being hollow or solid, and comprising the RTR component/s throughout the non-RTR solid particles, isotropically or anisotropically, homogeneously distributed or not, connected between them or not. These particles may be nanometric, micrometric, millimetric or macrosized.

It is also an object of the invention disclosed hereby to generate solid particles of environmentally responsive component/s, including, without limitation, RTR component/s, having any geometry, being hollow or solid, and comprising other RTR component/s throughout the first type of environmentally responsive solid particles, isotropically or anisotropically, homogeneously distributed or not, connected between them or not. These particles may be nanometric, micrometric, millimetric or macrosized. In another embodiment, the solid particles may dissolve over time contributing to the performance of the wound dressing.

In another embodiment, the RTR chains may be surface grafted to the substrate or any other component of the dressing by reacting the RTR chains or their precursors to functional groups present on the substrate surface layer or on the surface of any of the other component/s of the dressing, or by generating reactive anchoring sites on the surface of any of them by various techniques such as, without limitation, e-beam, UV or gamma radiation, chemical reactions and plasma treatments. This can be exemplified, without limitation, for plasma treatments, by exposing the wound dressing to plasma of ammonia, whereby amine moieties are generated on the surface of the wound dressing. These reactive groups perform then as anchoring sites for the RTR chains to bind to, via a coupling agent. Among other reactants, the substrate can be exposed to plasma of air, whereby various reactive groups, such as OH and COOH groups, are formed on the surface exposed.

In another embodiment, the RTR component/s may be thermoplastic or thermoset and combinations thereof and they may also be partially or totally biodegradable or non-biodegradable and combinations thereof.

It is also the object of this invention to generate multi-layered wound dressings, each layer with its own composition and properties, aimed to fulfill specific roles.

In another embodiment, a first layer formed by a suitable RTR polymer/s and on top of it, a second or more layer/s will be deposited in order to control other important properties of the wound dressing such as, and without limitation, optimize moisture transport, provide mechanical cushioning, and impart directionality to the diffusion of the biomolecules being released.

In another embodiment, the composition of the various components of this invention will be controlled not only along the z axis (from the interface with the tissue outwards) but also along the XY plane (from the center towards the periphery), to tailor other of its characteristics, for example, for forming a tighter seal on the healthy skin surrounding the wound or having the layer in direct contact with the wound having a higher concentration of active ingredients, among others.

It is also the object of this invention to allow direct visualization of the wound bed through the dressing.

It is also an embodiment taught by the present invention that the wound dressing is partially or totally in situ generated.

In another embodiment the RTR component/s may be additionally “programmed” so that their viscosity changes over time.

Below, follow a few examples, to briefly illustrate the invention disclosed hereby. The inventors have chosen to confine themselves to its application in the biomedical field, even though the compositions of the present invention can be applied to other areas.

Furthermore, for the sake of clarity and simplicity, and without limiting the scope of the invention in any form or fashion, the inventors have chosen to illustrate the invention hereby disclosed, by focusing on wound dressings and by exemplifying the invention using RTR polymers, and more precisely polymers comprising poly(ethylene oxide) and poly (propylene oxide) blocks. This, even though the compositions of the present invention include all families RTR polymers and also may be engineered to respond to other environmental stimuli. Focusing on a specific biomedical application and exemplifying the invention using one particular family of RTR polymers is intended only to illustrate preferred embodiments and should not be construed as limiting in any way or fashion, the scope of this invention, as more broadly set forth hereby.

It is an object of the invention to disclose a hydrogel wound dressings comprise one or more RTR components in low viscosity aqueous solution and one or more non-RTR components.

It is another object of the invention to disclose the wound dressing as defined above, wherein one or more RTR components are configured to generate said wound dressing either or both (i) in situ, namely at time of deploying said composition onto a body region under conditions allowing formation of a solid or semi-solid dressing including the formation of a film; and (ii) a priori such a deployment of said composition onto a body region; said conditions are selected from a group consisting of amount of material, speed of deployment, method of deployment, exposure to radiation, including UV, gamma radiation and plasma treatment, e-beam emission, chemical reactions including cross linker admixture, exposure to temperature, oxygen, and any combination thereof.

It is another object of the invention to disclose the wound dressing as defined above, wherein at least one RTR or layers or any other spatial arrangement thereof are interconnected between themselves and/or between one or more non-RTR components so as to form an integral part of said wound dressing.

It is another object of the invention to disclose the wound dressing as defined in any of the above, wherein at least one RTR component or layers or any other spatial arrangement thereof and at least one non-RTR component or layers or any other spatial arrangement thereof are not interconnected.

It is another object of the invention to disclose a biodegradable wound dressing as defined in any of the above, wherein the biodegradability is tunable in time or in space.

It is another object of the invention to disclose a biodegradable wound dressing as defined in any of the above, wherein at least one first component is configured to respond to at least one first stimulus and at least one second component to is configured to respond to at least one second stimulus, said stimulus is selected from a group consisting of temperature, time, water, oxygen concentration, NIR, IR, visible light or UV emission and any combination thereof.

It is another object of the invention to disclose a biodegradable wound dressing as defined in any of the above, wherein at least one component is configured to respond to one or more stimuli and release one or more components.

It is another object of the invention to disclose a biodegradable wound dressing as defined in any of the above, wherein at least one component comprises one or members of a group consisting of blends, semi-IPNs, IPNs, copolymers, derivatives, and any mixture or combination thereof.

It is another object of the invention to disclose a biodegradable wound dressing as defined in any of the above, wherein it further comprises at least one solid component that configured to appear in a diversity of shapes, sizes and geometries, including one or more members of a group consisting of spheres, particles of any other shape, capsules, fibers, ribbons, films, meshes, fabrics, non-woven structures, foams, porous structures of different types, each of them having the possibility of being solid, porous, hollow and combinations thereof and having a size spanning from nanometric to centimetric.

It is another object of the invention to disclose a biodegradable wound dressing as defined in any of the above, wherein it further comprises a component of relevance, said relevancy is selected from a group consisting of pharmacological and/or biological relevance, including drugs and drug residues, oligopeptide sequences, growth factors, material containing genetic information and combinations thereof.

It is another object of the invention to disclose a biodegradable wound dressing as defined in any of the above, wherein the component is blended with any other component of the wound dressing, prior to, during or after deployment.

It is another object of the invention to disclose a biodegradable wound dressing as defined in any of the above, wherein polymeric chains displaying the temperature-dependent coiling-uncoiling ability in aqueous media are attached to the surface of a substrate, typically polymeric.

It is another object of the invention to disclose a biodegradable wound dressing as defined in any of the above, wherein RTR are polymers selected from a group consisting of one or more members of a group consisting of poly(ethylene oxide)-poly(propylene oxide)-poly(ethylene oxide) (PEO-PPO-PEO) triblocks, random or alternating RTR PEO-PPO block copolymers, tetrafunctional block polymers of polyoxyethylene and polyoxypropylene condensed with ethylenediamine, N-alkyl substituted acrylamides (preferably poly-N-isopropyl acrylamide [PNIPAAm], cellulose derivatives, selected from a group consisting of hydroxypropyl methylcellulose and hydroxypropyl cellulose, alternating or random, and various amphiphilic polymers including poly(ethylene oxide)-polylactic acid block copolymers, and combination, mixture and derivative thereof.

It is another object of the invention to disclose a biodegradable wound dressing as defined in any of the above, wherein RTR chains are either or both surface grafted to the substrate or any other component of the dressing by reacting the RTR chains or their precursors to functional groups present on the substrate surface layer or any other spatial arrangement or on the surface of any of said other component/s of the dressing, or by generating reactive anchoring sites on the surface of any of them by various techniques selected from e-beam, UV or gamma radiation, chemical reactions and plasma treatments, or mechanically stabilized on the surface layer of the substrate by physical interlocking generating a surface confined blend, semi-IPN or IPN.

It is another object of the invention to disclose a biodegradable wound dressing as defined in any of the above, wherein RTR polymers comprises either or both poly(ethylene oxide) and poly (propylene oxide) blocks.

It is another object of the invention to disclose dressing, comprising at least one first RTR and an active component (AC) integrated within said RTR.

It is another object of the invention to disclose a biodegradable wound dressing as defined above, wherein AC is selected from at least one member of a group consisting of one or more INCI [i.e., cosmetics]; nutraceutical; medicament; cannabinoid, CBD, THC, cannabis and extracts thereof; household agent; agricultural agent; and industrial chemical agent.

It is another object of the invention to disclose a method of generating hydrogel wound dressings comprising providing one or more RTR components in low viscosity aqueous solution and one or more non-RTR components.

It is another object of the invention to disclose a method as defined above, wherein the method comprising steps deploying said one or more RTR components thereby generating said wound dressing, either or both (i) in situ, namely at time of deploying said composition onto a body region under condition allowing formation of a solid or semi-solid dressing including the formation of a film; and (ii) a priori said step of deploying of said composition onto a body region; said conditions are selected from a group consisting of amount of material, speed of deployment, method of deployment, exposure to radiation, including UV, gamma radiation and plasma treatment, e-beam emission, chemical reactions including cross linker admixture, exposure to temperature, oxygen, and any combination thereof.It is another object of the invention to disclose a method as defined in any of the above, wherein the method comprises step of interconnecting at least one RTR or layers or any other spatial arrangement thereof between themselves and/or between one or more non-RTR components under condition allowing formation of a film and wound dressing; said condition is one or more member of a group consisting of admixing a cross-linker; applying irradiation of the type, duration and intensity enabling interconnection; exposing to oxygen and any combination thereof.

It is another object of the invention to disclose a method as defined in any of the above, wherein at least one RTR component or layers or any other spatial arrangement thereof and at least one non-RTR component or layers or any other spatial arrangement thereof are not interconnected.

It is another object of the invention to disclose a method as defined in any of the above, wherein the method comprises step of tuning said biodegradability over time or to a defined space.

It is another object of the invention to disclose a method as defined in any of the above, wherein the method comprises step of configuring at least one first component to respond to at least one first stimulus and configuring at least one second component to respond to at least one second stimulus, said stimulus is selected from a group consisting of temperature, time, water, oxygen concentration, NIR, IR, visible light or UV emission and any combination thereof.

It is another object of the invention to disclose a method as defined in any of the above, wherein at least one component is configured to respond to one or more stimuli and release one or more components.It is another object of the invention to disclose a method as defined in any of the above, wherein the method comprises step of providing at least one component comprises one or members of a group consisting of blends, semi-IPNs, IPNs, copolymers, derivatives, and any mixture or combination thereof.

It is another object of the invention to disclose a method as defined in any of the above, wherein the method comprises step of shaping at least one solid component to shape, size and geometry being one or more members of a group consisting of 2D or 3D spheres of rounded cross sections, 2D or 3D polygonal of rectangular cross sections of any shape, capsules, fibers, ribbons, films, meshes, fabrics, non-woven structures, foams, porous hollow, continuous (i.e., non-porous) structures, being rigid, flexible semi-rigid and any combination, mixture or derivative thereof and having a size spanning from nanometric to centimetric.

It is another object of the invention to disclose a method as defined in any of the above, wherein the method comprises step of providing the wound dressing with one or more components of relevance, said relevancy is selected from a group consisting of pharmacological and/or biological relevance, including drugs and drug residues, oligopeptide sequences, growth factors, material containing genetic information and combinations thereof.

It is another object of the invention to disclose a method as defined in any of the above, wherein the method comprises step of blending said component with any other component of the wound dressing, prior to, during or after deployment.

It is another object of the invention to disclose a method as defined in any of the above, wherein the method comprises step of configuring polymeric chains to display a temperature-dependent coiling-uncoiling ability in aqueous media are attached to the surface of a substrate, typically polymeric.

It is another object of the invention to disclose a method as defined in any of the above, wherein the method comprises step of selecting said RTR from one or more members of a group consisting of poly(ethylene oxide)-poly(propylene oxide)-poly(ethylene oxide) (PEO-PPO-PEO) triblocks, random or alternating RTR PEO-PPO block copolymers, tetrafunctional block polymers of polyoxyethylene and polyoxypropylene condensed with ethylenediamine, N-alkyl substituted acrylamides (preferably poly-N-isopropyl acrylamide [PNIPAAm], cellulose derivatives, selected from a group consisting of hydroxypropyl methylcellulose and hydroxypropyl cellulose, alternating or random, and various amphiphilic polymers including poly(ethylene oxide)-polylactic acid block copolymers, and combination, mixture and derivative thereof.

It is another object of the invention to disclose a method as defined in any of the above, wherein the method blend, semi-IPN or IPN.

comprises step of configuring RTR chains to be either or both surface grafted to the substrate or any other component of the dressing by reacting the RTR chains or their precursors to functional groups present on the substrate surface layer or any other spatial arrangement or on the surface of any of said other component/s of the dressing, or by generating reactive anchoring sites on the surface of any of them by various techniques selected from e-beam, UV or gamma radiation, chemical reactions and plasma treatments or mechanically stabilized on the surface layer of the substrate by physical interlocking generating a surface confined

It is another object of the invention to disclose a method as defined in any of the above, wherein the method comprises step of providing the RTR polymers to comprise either or both poly(ethylene oxide) and poly (propylene oxide) blocks.

It is another object of the invention to disclose a method of providing a dressing by providing both (i) at least one first RTR and (ii) at least one active component (AC) integrated within said RTR.

It is another object of the invention to disclose a method as defined above, wherein the method comprises step of comprising steps deploying said at least one first RTR components thereby generating said dressing, either or both (i) in situ, namely at time of deploying said composition onto a predefined surface under condition allowing formation of a film; and (ii) a priori said step of deploying of said composition onto said surface; said conditions are selected from a group consisting of amount of material, duration of exposure, temperature, exposure to oxygen, UV and a combination thereof.

It is another object of the invention to disclose a method as defined above, wherein AC is selected from at least one member of a group consisting of one or more INCI [i.e., cosmetics]; nutraceutical; medicament; cannabinoid, CBD, THC, cannabis and extracts thereof; household agent; agricultural agent; and industrial chemical agent.

It is another object of the invention to disclose a method of treating a medical or cosmetic indication by either or both (i) in situ generating a wound dressing, and a priori generating said wound dressing; said step of in situ generating of said wound dressing is provided at time of deploying said composition onto a predefined surface under condition allowing formation of a film; said step of a priori generating of said wound dressing is provided before said step of deploying of said composition onto said surface; said conditions are selected from a group consisting of amount of material, exposure to radiation, including UV, gamma radiation and plasma treatment, e-beam emission, chemical reactions including cross linker admixture, exposure to temperature, oxygen, and any combination thereof.

It is another object of the invention to disclose a method as defined above, wherein the aforesid indication is selected from a group consisting of vaginal, anal, rectal, proctology, dermal, buccal, growth factors, H acid, trichology, NACS/NSID (anti-inflammatory), ontological preparation (EARS plug), anti-fungal (chlorohexidine), sun screens UV blockers, anti-aging serum, mucosal preparations, veterinary products.

It is another object of the invention to disclose a method of treating a medical or cosmetic indication by providing a dressing with at least one first RTR and with at least one active component (AC) integrated within said RTR.

It is another object of the invention to disclose a method as defined above, wherein the method comprises step of deploying said at least one first RTR components thereby generating said dressing, either or both (i) in situ, namely at time of deploying said composition onto a predefined surface under condition allowing formation of a film; and (ii) a priori said step of deploying of said composition onto said surface; said conditions are selected from a group consisting of amount of material, exposure to radiation, including UV, gamma radiation and plasma treatment, e-beam emission, chemical reactions including cross linker admixture, exposure to temperature, oxygen, and any combination thereof.

It is another object of the invention to disclose a method as defined above, wherein the aforesaid indication is selected from a group consisting of vaginal, anal, rectal, proctology, dermal, buccal, growth factors, H-acid, trichology, NACS/NSID (anti-inflammatory), ontological preparation (EARS plug), anti-fungal (chlorohexidine), sun-screens UV blockers, anti-aging serum, mucosal preparations, and veterinary products.

It is another object of the invention to disclose kit for the preparation and administration of a hydrogel wound dressings. The kit comprises means, such as at least two sealable, single or multiple compartment syringes, configured b means of volume and tip-shape to store and deploy one or more RTR components in low viscosity aqueous solution and one or more non-RTR components.

It is another object of the invention to disclose kit as defined above, wherein the kit comprises means for generating conditions for film formation, said means are selected from a group consisting of means for deploying an effective amount of material, radiation source for exposing either or both aforesaid RTR and non-RTR components to an effective radiation dose, including UV radiation, gamma radiation and plasma treatment, e-beam emission, means for admixing and thereby providing an effective chemical reaction of either or both aforesaid RTR and non-RTR components and cross linker, heater or cooler for exposing the same to a predefined temperature, supplier of oxygen, and any combination thereof.

It is another object of the invention to disclose the kit as defined in any of the above, the one or more RTR components are configured to generate said wound dressing either or both (i) in situ, namely at time of deploying said composition onto a body region under condition allowing formation of a film; and (ii) a priori such a deployment of said composition onto a body region; said conditions are selected from a group consisting of amount of material, exposure to radiation, including UV, gamma radiation and plasma treatment, e-beam emission, chemical reactions including cross linker admixture, exposure to temperature, oxygen, and any combination thereof.

Furthermore, while the invention will now be described in connection with certain preferred embodiments in the following examples and with reference to the attached figures so that aspects thereof may be more fully understood and appreciated, it is not intended to limit the invention to these particular embodiments. On the contrary, it is intended to cover all alternatives, modifications and equivalents as may be included within the scope of the invention. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### BRIEF DESCRIPTION OF THE DRAWINGS

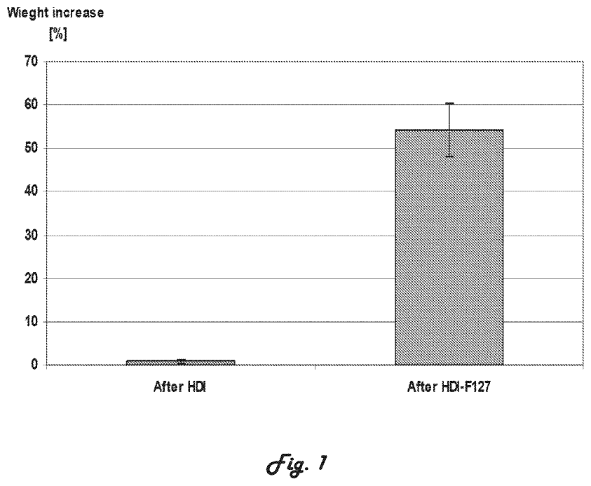

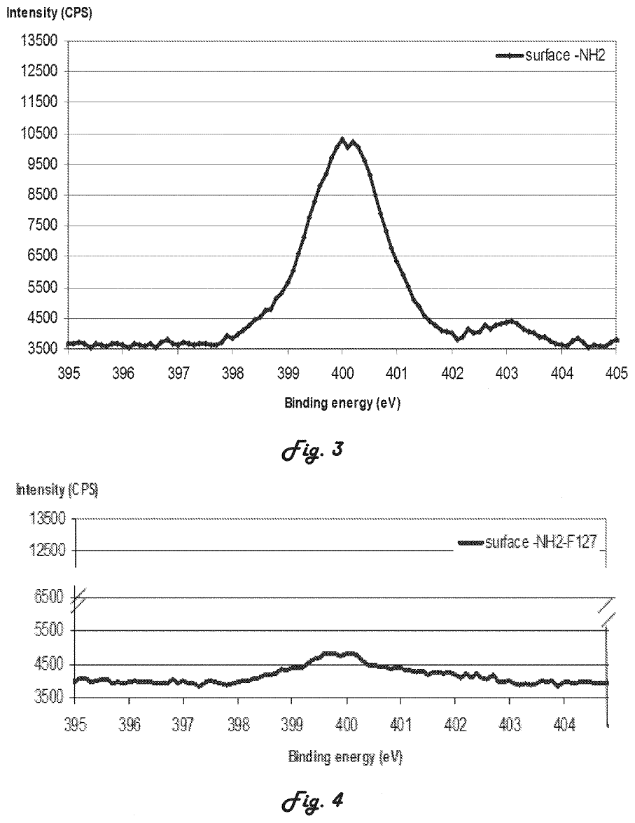

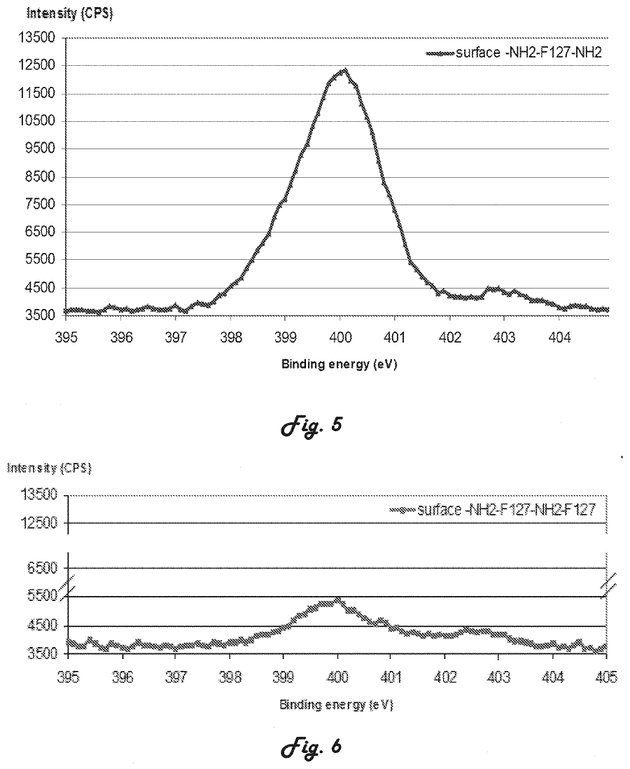

The accompanying drawings, which are incorporated into and form a part of the specification, illustrate several embodiments of the present invention and, together with the description, serve to explain the principles of the invention. The photos, schemes, graphs and drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention, in which FIGS. 1 to 44 depict certain embodiments as described herein.

### DETAILED DESCRIPTION OF THE PREFERRED EMBODIMENTS

Some of the embodiments of the invention disclosed hereby will be exemplified using the family of polymers comprising poly(ethylene oxide) (PEO) and poly(propylene oxide) (PPO) chains. These can be part of di or triblocks, such as poly(ethylene oxide)-poly(propylene oxide)-poly(ethylene oxide) (PEO-PPO-PEO) triblocks, commercially available as Pluronic©, and primarily, {EO}₉₉-{PO}₆₇-{EO}₉₉, known as Pluronic F127. They could also be part of other molecules comprising these two types of polyether chains, such as, without limitation, tetra-armed molecules.

Also, high molecular weight RTR polymers produced by covalently binding PEO-PPO-PEO triblocks using reactive bifunctional molecules such a diisocyanates, diacyl chlorides, phosgene, among others, were used as well. Among them, hexamethylene diisocyanate (HDI), was typically used. Additionally, block polymers consisting of poly(ethylene oxide) (PEO) and poly(propylene oxide) (PPO) segments, coupled via diverse coupling agents, such as diisocyanates, diacyl chlorides, phosgene, among others, were used as well. Other chemistries such as Michael addition, thiol-ene and click chemistry mechanism may be used, among numerous others, as required.

[a] Synthesis of PF-127. Pluronic F-127 (molecular weight 12,600) was poured in a three-necked flask and dried. Then, the corresponding amount of HDI and SnOct₂ (0.64 wt %) were added to the reaction mixture and reacted at 80° C. for 30 minutes under mechanical stiffing (160 RPM) and dry nitrogen atmosphere. The polymer produced was dissolved in chloroform and precipitated in a petroleum ether 40-60 ethyl ether mixture (1:1). Finally, the polymer was washed repeatedly with portions of petroleum ether and dried. Different F-127/HDI ratios resulted in different degrees of polymerization (DP).

[b] Synthesis of Poly(ether-carbonate)s. These polymers were synthesized by copolymerizing poly(ethylene glycol) and poly(propylene glycol) segments utilizing

phosgene as the coupling molecule. The different reactivity of phosgene's two functionalities allowed binding the two constituents, in both an alternate or random mode.

The synthesis of the alternating poly(ether-carbonate)s was carried out following a two-step reaction, as described elsewhere in detail. The first step was the PEO dichloroformate synthesis, followed by the reaction between the PEG derivative and the PPG chain, to produce the final block copolymer. The random poy(ether-carbonate)s were synthesized by a similar one-pot reaction, as described elsewhere in detail.

[c] Synthesis of Poy(ether-ester)s. The synthesis is exemplified hereby for a copolymer containing PEO6000 and PPO3000 segments. Equimolar amounts of dry PEG6000 and dry PPG3000 were dissolved in 30 ml dry chloroform in a 250 ml flask.

Triethylamine (2:1 molar ratio to PEG) was added to the reaction mixture, followed by the dropwise addition of the diacyl chloride (2:1 molar ratio to PEG) in dry chloroform over a period of 30 minutes at 40oC, under magnetic stiffing. Then, the temperature was risen to 60oC and the reaction was continued for additional 90 minutes. The polymer produced was separated from the reaction mixture by adding to it about 600 ml petroleum 14 ether 40-60. The lower phase of the two-phase system produced was separated and dried at RT. Finally, the polymer was thoroughly washed with petroleum ether and dried. Light yellow, brittle and water soluble powders were obtained.

[d] Synthesis of Poy(ether-ester-carbonate)s. The synthesis is exemplified hereby for a copolymer containing PEO6000 and PPO3000 segments, caprolactone blocks comprising four repeating units and phosgene. The (CL)₄-PEO6000-(CL)₄ triblock was synthesized as follows: 30.3 g of PEG6000 were dried at 120 ° C. under vacuum for 2 hours. Then, 10.1 g ε-caprolactone and 0.05 g stannous 2-ethyl-hexanoate were added. The reaction mixture was heated at 145oC for 2.5 hours in a dry nitrogen atmosphere.

Finally, the reaction mixture was cooled to room temperature, dissolved in chloroform, precipitated in petroleum ether and dried at room temperature. Once the (CL)₄-PEO6000-(CL)₄ triblock was obtained, the reaction with phosgene and the final reaction with the PPG chain were performed as described above.

In some embodiments, the invention disclosed hereby comprises crosslinked RTR components. The synthesis of some of these crosslinked gels is presented below for illustration purposes only, without limiting the invention in any way or manner.

[e] Synthesis of PEO-PPO-PEO dimethylmethacrylate. The reaction is shown for F127, even though it was conducted also for other PEO-PPO-PEO triblocks, such as F-87. 40.1 g (3.2 mmol) of Pluronic F127 were poured in a three-neck flask and dried as described above. Then, the polymer was dissolved in 75 ml of dry chloroform and the solution was cooled to 0° C. in an ice bath. 2.63 g of TEA (26.3 mmol) were added. 2.65 g (26 3 mmol) of freshly distilled methacryloyl-chloride were diluted in 20 ml chloroform and added dropwise for 2 hours into the cooled mixture, under dry nitrogen flow and magnetic stiffing. Finally, the reaction was allowed to proceed for 24 hours at room temperature. The crude product was dried under vacuum and was re-suspended in hot toluene (100 ml). The hot mixture was then filtered in order to remove the triethylammonium hydrochloride salt. The toluene solution was precipitated in 400 ml of petroleum ether 60-80° C. The white solid product, Pluronic F127 dimethacrylate (F127-DMA), was filtered under vacuum, washed with several portions of petroleum ether 40-60° C. and dried under vacuum at room temperature.

The functionalized triblocks will be denoted F127-DMA and F87-DMA, respectively.

Preparation of crosslinked F127-DMA gels. The crosslinking reaction is hereby exemplified for F127-DMA, even though it was conducted also for F87-DMA. The gels were prepared by dissolving 3 g of F127-dimethacrylate in 12 ml of distilled water. 20 mg of ammonium persulfate (APS) were dissolved in 100 μl water and added to the solution at low temperature and homogenized. Then, 20 mg of sodium metabisulfite were dissolved in 100 μl water, added to the solution and mixed thoroughly. Finally, the 20% (wt/wt) polymer system was incubated at 37° C. for 24 hours. The crosslinked gels will be denoted X-F127 and X-F87.

The formation of F-DMA molecules was also achieved by reacting the triblocks with isocyanate ethyl methacrylate (IEMA), under conditions suitable to that reaction.

Preparation of crosslinked F127-DIPTS. Ethoxysilane-capped Pluronic F127 (F127-DIPTS). 25.2 g (0.002 mol) of Pluronic F127 were weighed in a three-necked flask and dried as described above. Then, 1.2 g (0.005 mol) IPTS and 0.1 g (3.10-4 mol) SnOct₂ were added to the reaction mixture and reacted at 75° C. for one hour, under mechanical stiffing and a dry nitrogen atmosphere. The material produced was dissolved in chloroform (30 ml), precipitated in petroleum ether 40-60 ° C. (400 ml) and filtered.

Finally, the F127 derivative (F127-DIPTS) was washed repeatedly with portions of petroleum ether 40-60 ° C. (3×100 ml) and dried in vacuum at room temperature.

The switchable surfaces of this invention covered different types of RTR chains and were prepared following diverse strategies, differing, without limitation, in the mechanism used to anchor the chains to the substrate. The latter is exemplified below for two of the various approaches followed: [i] Covalently bound and [ii] Physically entrapped.

### [A] Covalently Bound

The different grafting schemes used require the presence of reactive functional groups on the surface of the wound dressing, for them to perform as reactive anchoring sites for the covalent binding of the RTR moieties. These binding sites can be part of the polymer present, for example, as pendant groups along the backbone. The groups can be, among others, and without limitation, hydroxyl, carboxylic, amine, nitrile, ester, amide, thiol and isocyanate, and carbon double bonds. These binding sites can be generated on the surface by a diversity of techniques, such as hydrolyzing the surface by exposing if to suitable reagents, such as NaOH, among others, or other chemicals, or various types of radiation.

The case where the invention disclosed hereby capitalized on “autologous” functional groups is exemplified below for Nylon yarns having amine groups on their surface. These functionalities were reacted with HDI by immersing the yarns in HDI at 37 ° C. for 30 minutes, and after thoroughly rinsing them to remove all non-reacted HDI that remained adsorbed on the surface, they were reacted with F127, in the presence of the SnOct catalyst.

FIG. 1 presents the weight increase shown by the fibers after surface reacting with HDI only, and after completing the reaction with the F127.

The weight increase shown by the Nylon fibers after reacting with HDI and after reacting first with HDI and then with F127.

The fact that the F127 chains are covalently bound to the surface was demonstrated as follows: fibers that were not reacted first with HDI, did not show any weight increase, after trying to reacting them with the triblock, under the same reaction conditions.

Plasma was one of the techniques used to create reactive anchoring site on the surface of the wound dressing, so that the RTR chains can be covalently bind to it. Plasma is a powerful and versatile technique which is able to dramatically alter the surface chemical composition of substrates, without affecting their bulk properties. The modification of the surface of the wound dressing can be affected by various processes which, by adding, abstracting or rearranging surface species, result in the functionalization, etching or crosslinking of the material's surface layer. In one of the plasma treatments used, the wound dressing was exposed to plasma of ammonia, which generated amine groups, covalently bound to the surface of the substrate. Even though other conditions were used, typically the plasma system functioned at 600 mTorr and 25 W, for 8 minutes. Subsequently, the just generated amine groups were reacted with various multifunctional molecules, containing two or more reactive groups, with one group reacting with the surface amine and the rest performing as anchoring sites for further derivatization with the thermo-responsive chains. This is illustrated below for hexamethylene diisocyanate, which reacts with the amine present on the surface of the wound dressing, whereby urea moieties are formed, while the second NCO groups remain reactive and bind the thermo-responsive chains via their hydroxyl end groups This is illustrated below for F127 chains.

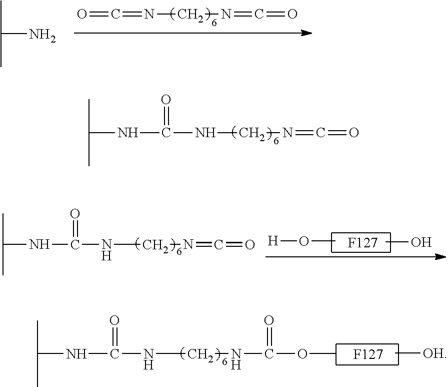

The actual occurrence of the various reactions and the composition of the new surfaces created at the different stages of the tailoring process were demonstrated by XPS, FTIR spectroscopy and contact angle studies. This is exemplified hereby, without limitation, for a Dacron {poly(ethylene terephthalale), PET} substrate. The XPS spectrum of untreated PET showed the carbon and oxygen peaks only, the 2.43 (C/O) ratio measured being very close to the theoretical 2.50 value. These findings are indicative of an essentially uncontaminated substrate surface. The characteristic C1s peaks at 285.0 eV, 286.4 eV and 288.9 eV, assigned to the aliphatic, ether and ester carbon atoms, respectively, as well as the Oxygen-1s peaks at 532.0 and 535.5 eV, were clearly seen. Substantial amounts of nitrogen were found on the surface, after exposing the substrate to plasma of ammonia, as revealed by a large N1s peak at 402.0 eV. Expectedly, the low resolution XPS spectrum of this new surface showed also the C1s and the peaks due to the Dacron substrate. The amine groups were then reacted with hexamethylene diisocyanate (HDI), as a result of which urea bonds were obtained, while the remaining free isocyanate group served as a reactive site for further derivatization. The last reaction of the surface modification scheme was the grafting of PEO-PPO-PEO triblocks, as shown in the scheme above, by reacting their hydroxyl terminal group with the NC0 functionality on the surface. The presence of the triblocks was demonstrated by the C1s spectrum which showed the sharp ether peak centered at 286.4 eV. Furthermore, the appearance of the large C1s ether peak was accompanied by a large increase in the O1s peak due to the ether oxygen seen at 531.6 eV.

Aiming at gaining further insight into the nature of the surfaces generated, FTIR spectroscopy analysis was conducted at the different stages of the process. PET exhibited its characteristic bands at 1740 cm⁻¹, due to the stretching of the ester carbonyl group, and at 1100 cm⁻¹, belonging to the stretching of the C—O—C bond. The presence of the amine groups introduced by the ammonia plasma could not be detected directly due to the limited surface sensitivity of this technique, but their presence was demonstrated indirectly (see below). The FTIR spectra of PET after being exposed to the plasma treatment, and then following the reaction with HDI, first, and then with PEO-PPO-PEO chains, demonstrated the occurrence of the reaction and the covalent nature of the binding between the different steps of the tailoring scheme. Given the conditions of the system, the reaction of the HDI molecules with the amine groups takes place through only one of its isocyanate groups, leaving the second one available for further derivatization. The FTIR spectrum of PET-NH₂, after reacting with HDI and thoroughly rinsing off unbound molecules, showed the characteristic peak of the free isocyanate group at 2272 cm⁻¹, indicating the presence of the HDI molecules. Furthermore, the sharp peak at 1640 cm⁻¹ present is attributed to the urea bond created by the reaction between the amine group and HDI. The last stage of the derivatization process capitalized on the presence of the free isocyanate groups by reacting them with a hydroxy-terminated PEO-PPO-PEO triblocks.

The characteristic peak of the isocyanate group at 2272 cm⁻¹ disappeared, indicating that the isocyanate groups reacted with the F127 molecules. The significant increase in the 19 ether absorbance band at 1110 cm⁻¹ is also indicative of the presence of the PEO-PPOPEO chains bound to the substrate's surface. To conclusively demonstrate that the HDI and PEG molecules were covalently bound to the surface and not just adsorbed onto it, control experiments were conducted. For example, when the PET surface was directly put in contact with HDI, without previously introducing amine groups into the surface, even an extremely mild rinsing procedure was successful in removing almost completely the adsorbed HDI molecules. Also, the urea linkage, absorbing at 1640 cm⁻¹, generated by the amine-isocyanate reaction, could not be found in the spectrum of the control sample.

Fully consistent with the already described XPS data, these findings provide supporting evidence, even though indirectly, proving the presence of amine groups on the surface, following its exposure to plasma of ammonia. In striking contrast to this behavior, the amine-containing surface readily reacted with HDI, the presence of the second NCO group being evident from the large peak centered at 2272 cm⁻¹. Also, the sharp urea band at 1640 cm⁻¹ was now readily seen. A similar experiment was conducted, aiming at shedding some light on the nature of the binding of the PEO-PPO-PEO triblocks to the surface. In this case, no intermediate HDI molecule was grafted to the surface, and the F127 chains were reacted directly to the amine containing surface. Expectedly, no reaction took place, the PEO-PPO-PEO chains being readily rinsed off by an extremely mild procedure.

The water contact angle of the untreated PET film was between 70° and 74° , decreasing drastically after the incorporation of the amine groups into the surface, while an increase to around 60° being apparent, after grafting the PEO-PPO-PEO triblocks.

The length of the RTR chains grafted onto the surface of the wound dressing was anticipated to significantly enhance their ability to coil and uncoil and, in turn, to generate more effective “detachable” interfaces with the wound. Thus, also longer multiblock polymers, generated by the polymerization of Pluronic F127 using hexamethylene diisocyanate (HDI) as the chain extender, were grafted onto the wound dressings. Alternatively, also, a multistep scheme, we named “The Wedding Cake” Model, was developed, whereby after the first PEO-PPO-PEO chains were grafted onto the surface of the wound dressing. The hydroxyl end group of the first triblocks were subsequently reacted with HDI, for example, and then the remaining reactive NCO groups was reacted, in turn, with yet another PEO-PPO-PEO triblock, via its terminal OH group. This process was repeated several times, generating, therefore, long RTR chains. Also here, each of the steps of the process was studied and the covalent nature of the binding between each of the consecutive steps was demonstrated.

Aiming at maximizing the efficiency of the grafting scheme, a multi-stage scheme, sequentially combining repeated exposure to plasma of ammonia and subsequent

reactions using HDI and F127, was performed. More specifically, after the first layer of PEO-PPO-PEO chains was surface grafted as described above, then it was exposed to plasma of ammonia, creating a number of amine functionalities along F127's backbone, which, in turn, were reacted to additional triblocks via HDI spacers.

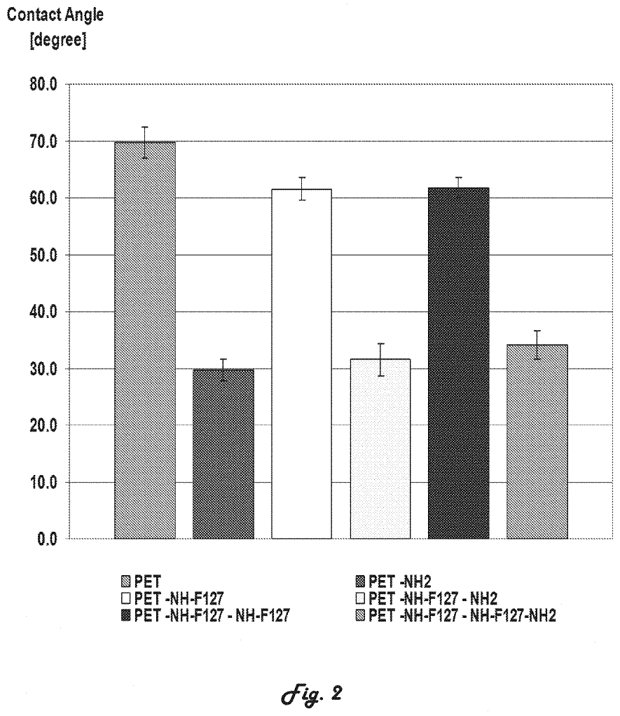

Reference is now made to FIG. 2. While in the first stage of this surface modification scheme, an amine-to-F127 ratio of 1 is obtained, this ratio increases remarkably during the next step, and even more so, during the third one, and so forth. It is apparent, therefore, that this step-by-step process produced dendrimer-like, surface grafted arborescent RTR structures, expected to be rather effective. Due to its nature, the new surface later, interfacing with the tissue bed was named “The Afro Model”. The different steps of the process were characterized as done with the other strategies, using XPS and contact angle analyses, among other techniques, such as SEM studies. Water contact angle measurements performed at the different stages of the process, revealed the fluctuations in hydrophilicity, as the outer layer of the yarn varied, as shown below. In agreement with theoretical considerations, amine-rich surfaces were the most hydrophilic, with contact angles of 32-35 degrees, while the PEO-PPO-PEO triblocks resulted in relatively more hydrophobic surfaces, with contact angles around 60 degrees.

The occurrence of the different grafting reactions was demonstrated using diverse techniques, including XPS spectroscopy, water contact angle measurements (on model surface). Reference is now made to FIGS. 3-6, presenting N 1 s XPS spectrum for each of the steps, clearly demonstrating the occurrence of each of the various steps of this advanced grafting scheme.

Additional embodiments of the present invention were also engineered and their composition, covalent nature and RTR behavior were demonstrated.

New RTR polymers systems combining RTR behavior and displaying gradually increasing mechanical properties over time, were created by crosslinking silane-capped poly(ethylene oxide)-poly(propylene oxide)-poly(ethylene oxide) triblocks in aqueous solutions at physiological conditions. Pluronic F127 (PEO₉₉-PPO₆₇-PEO₉₉) was functionalized with (3-isocyanatopropyl) triethoxysilane (IPTS) by reacting its terminal hydroxyl groups with the isocyanate. The silane-capped PEO-PPOPEO triblock was characterized by ¹H-NMR, GPC, FT-IR and DSC and the rheological behavior of its aqueous solutions were studied. The silane-containing triblock retained the reverse thermo-responsive characteristics displayed by the original Pluronic. Over time, the ethoxysilane groups hydrolyzed and created silanol moieties that subsequently condensated, crosslinking the material and generating hydrogels that exhibited gradually increasing mechanical properties. It was found that the higher the pH, the faster the process and the higher the viscosity levels attained. Also, the ability of these gels to perform as matrices for drug delivery was exemplified by releasing metronidazole and methylene blue. Findings showed that while a 30% F127 gel at 37° C. delivered all the drug within less than 3 days, F127di-IPTS gels completed the process at a much slower rate (up to 15 days).

This strategy was used not only when the RTR component of the invention disclosed hereby was used separately from the wound dressing itself, as described above, but it was also harnessed to generating a covalently bound RTR interface with the wound dressing. One of the embodiments taught by this invention combined the plasma treatments described above, and the reactivity of the amine-isocyanate reaction. In this case, after exposing the wound dressing to plasma of ammonia, the amine moieties generated on the surface of the substrate were reacted with (3-isocyanatopropyl) triethoxysilane (IPTS). Then, once in contact with water and in the presence of IPTS-capped PEO-PPO-PEO triblocks, the the ethoxysilane groups hydrolyzed and created silanol moieties, that subsequently condensated, crosslinking the material and generating surface grafted thermo-responsive hydrogels. This approach has two additional advantageous features. One, is that it easily allows to generate thicker surface grafted thermo-responsive layers, and also that the amine groups initially generated on the surface allow the binding of more than one RTR layer.

This was also achieved by reacting the amine group to tri-isocyanates, such as isocyanurate tri-isocyanate rings, whereby one NCO group reacted with the amine group generated by the plasma of ammonia, and the other two reacted with F127 chains via their hydroxyl end groups. This scheme was repeated several times, generating a surface grafted RTR dendrimeric construct. Additionally, instead of using a triisocyanate, HDI and other diisocyanates were used, including isocyanate-terminated F127 chains, were reacted with the initial amine, and then the remaining reactive NCO moiety was reacted with a multi-functional molecule, such as triols or triamines, or molecules containing others functionalities capable of reacting with the NCO group, and having more than three reactive groups. They may also comprise different groups such as, without limitation, one amine and two hydroxyl groups, among many other combinations. Also, oligopeptides containing various reactive groups can be used.

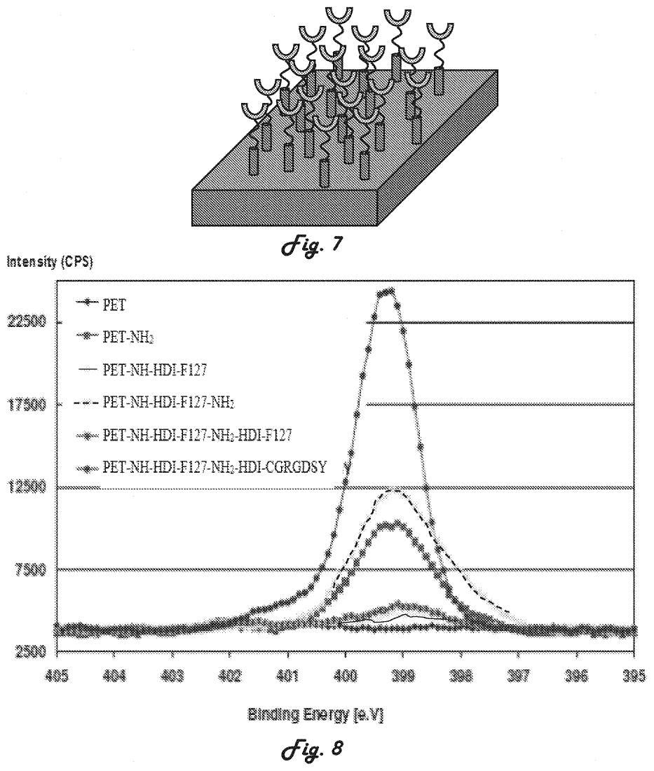

In some instances also biologically active molecules were added to the systems, by just blending them into one or more components of the wound dressing, including the RTR component. In other instances, it was covalently bound to the RTR component, generating an interface as described in FIG. 7.

Reference is now made to FIG. 8 which presents spectra of a PET substrate, where several layers of PEO-PPO-PEO triblocks were grafted, and finally end grafted, distally to the wound dressing, with an oligopeptide. In this case, an RGD-containing motif was reacted via the thiol end group of the terminal cysteine amino acid of the oligopeptide sequence, to an NCO-capped, surface grafted F127 chain.

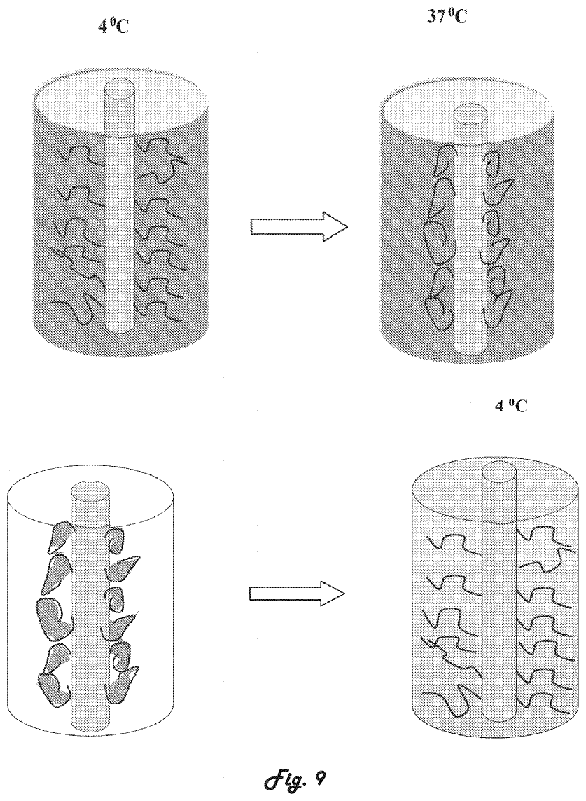

The ability of the surface grafted RTR chains to coil and uncoil was demonstrated also by entrapping model molecules, mimicking drugs or other molecules of biological relevance, within the surface grafted chains. FIG. 9 schematically describes the working concept pursued, where the RTR chains bound to the substrate, coil and uncoil in an aqueous medium, as a function of temperature. They are uncoiled at low temperature and the model molecules (Methylene Blue) then diffuse among the chains. Subsequently, the wound dressing is applied to the wound and upon its contact with the tissue, the chains coil due to the increase in temperature, entrapping the drug, and releasing it over time, later on, typically at the temperature at the site.

Furthermore, an “on command” strategy” is easily implemented, whereby the wound dressing is cooled down in a controlled manner, so to allow the faster release of enhanced doses of the drug, at specific time points, as required clinically.

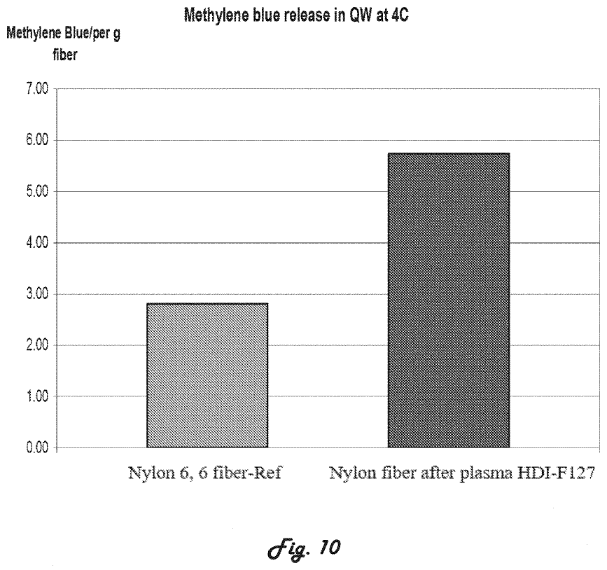

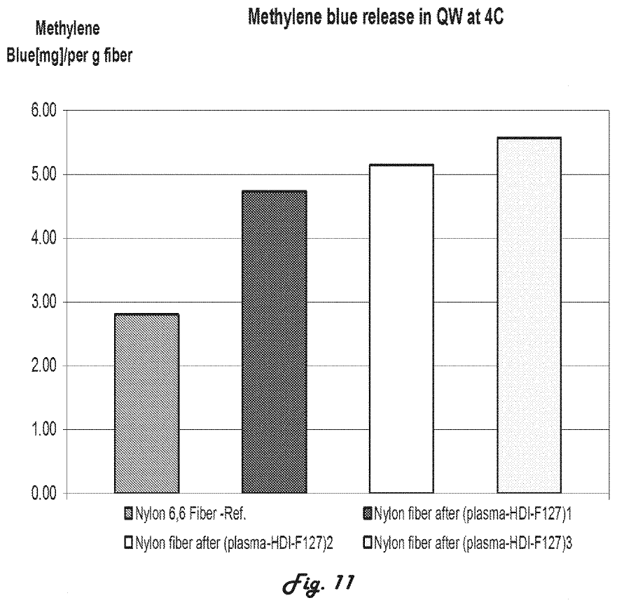

The amount of Methylene Blue entrapped on the surface of the fibers is presented in FIG. 10, in this case for a Nylon yarn, after being unloaded in cold water (4° C.), and compared to the amount adsorbed on the surface of untreated yarns, due to non-specific adsorption. Amount of Methylene Blue unloaded from treated and untreated Nylon fibers.

The data presented in FIG. 11 illustrate the effect of additional layers of RTR chains, as per the sequential grafting scheme described above on the ability of the yarns to entrap increasing amounts of cargo.

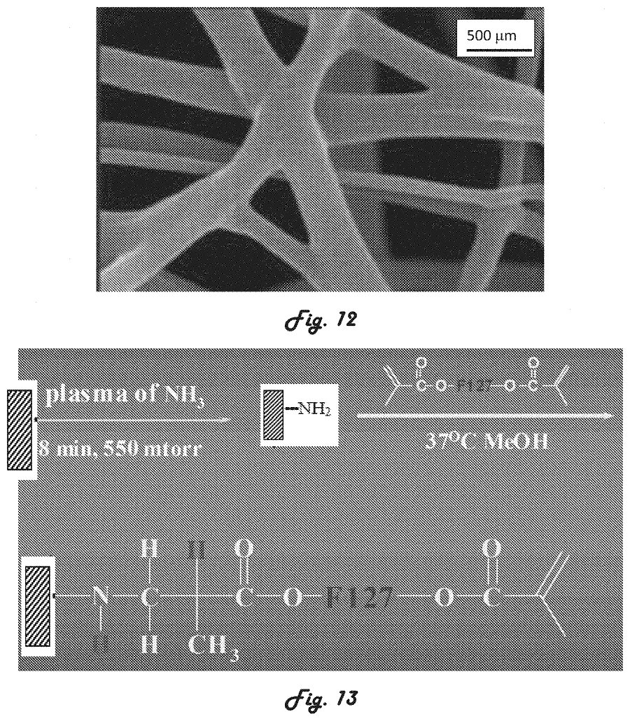

Amount of Methylene Blue unloaded from treated yarns with one, two and three layers of F127, and untreated Nylon fibers. The surface grafted layers described hereby, can be grafted on substrates of a broad range of sizes, from macroscopic, to micrometric, and down to fibers having diameters in the nanoscale, like the ones showed in FIG. 12.

An additional embodiment of the invention disclosed hereby, describes the reaction of the RTR chain with the plasma generated amine groups, capitalizing on the Michael Addition reaction. In these embodiments, the amine groups on the surface of the wound dressing are reacted with the double bond incorporated into properly functionalized RTR chains. This is exemplified hereby, without limitation, for F127 dimethacrylate chains, as shown below. The F127 dimethacrylates are typically formed by reacting the terminal OH groups of the PEO-PPO-PEO triblocks with acryloyl chloride or isocyanate ethyl dimethacrylate (IEMA).

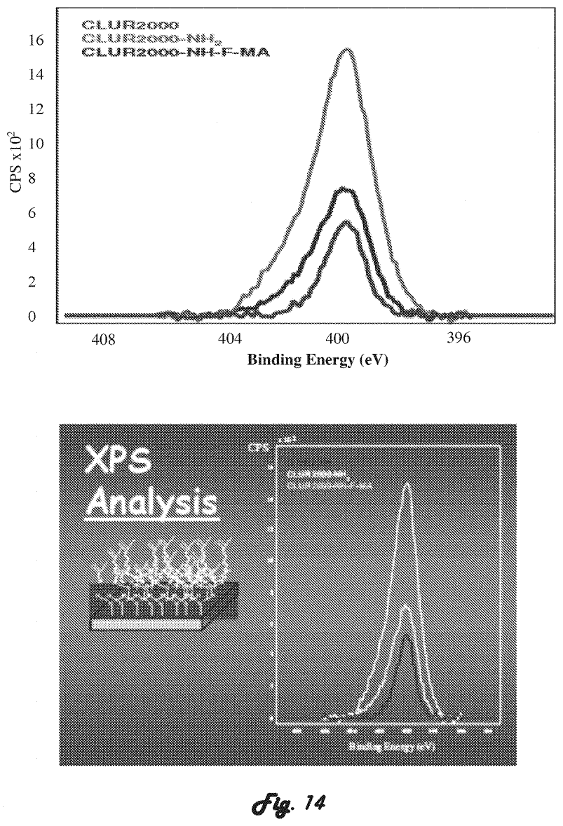

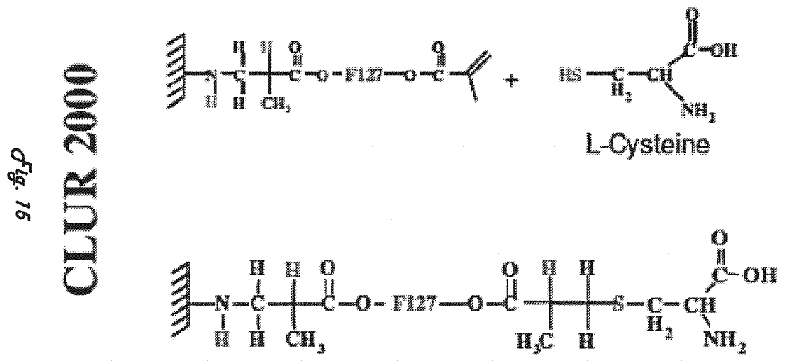

This grafting scheme is shown in FIG. 13, when applied to a degradable polymer comprising polycaprolactone segments having a molecular weight of 2000, coupled together with HDI, whereby amide groups are formed along the backbone. F127 dimethacrylate was reacted with the amine groups, are shown in FIGS. 14.

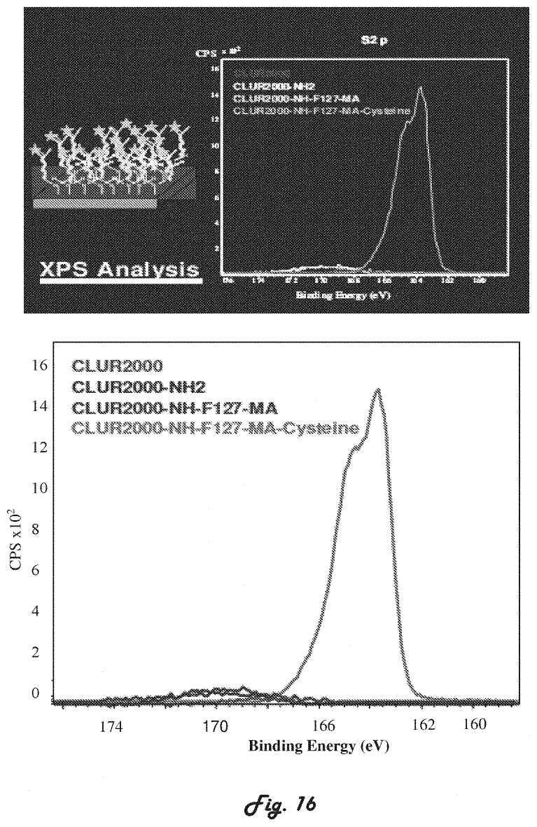

In some embodiments, the unreacted double bond is reacted with other species, such as, and without limitation, molecules of biological importance. In FIG. 15, this is exemplified for cysteine, where its thiol group reacts with the double bond, covalently binding this amino acid to the wound dressing. The occurrence of the reacting is conclusively demonstrated by XPS, as can be seen in FIG. 16.

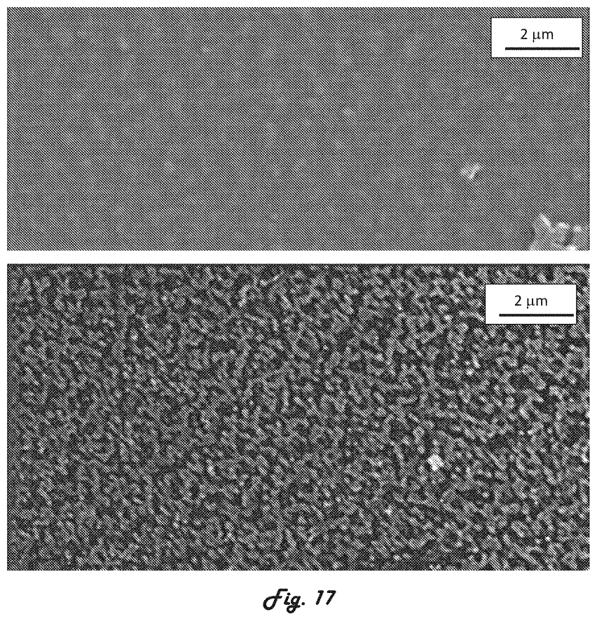

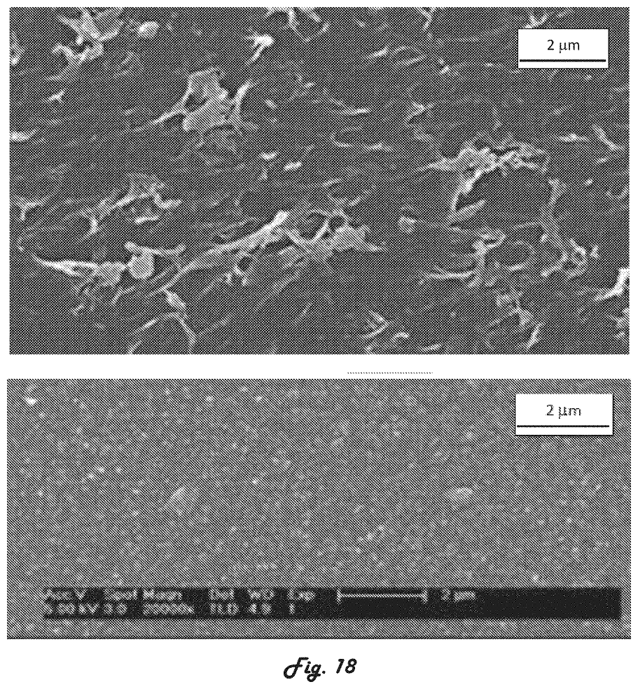

The SEM micrographs shown in FIG. 17-18 present the surface of untreated PET and PET after being exposed to plasma of ammonia and being grafted with F127 dimethacrylate. The profound difference between the two surface is evident.

Then, the second double bond, still present after the grafting reaction, was used to crosslink the surface grafted chains. Furthermore, the chains where crosslinked while in an aqueous medium at two temperatures, one above and one below the relevant thermal transition. The surfaces generated are hence presented.

In accordance with theoretical considerations, the RTR chains were crosslinked while being coiled or expanded, depending on the temperature at which the crosslinking reaction was conducted. The thermo-responsiveness of the new surfaces is apparent from the different morphologies displayed by the surfaces produced.

Thicker RTR layers were formed on the wound dressing's surface. Furthermore, aiming at generating a new, efficient, safe and detachable wound dressing-tissue interface, in some embodiments of this invention, a concentration gradient was engineered, by varying various experimental and compositional parameters. The objective here was to combine (a) robustness of the attachment of the RTR component to the rest of the dressing and (b) maximum chain mobility at the very interfacial layer between the RTR layer of the wound dressing and the wound. Since chains attached to the surface of the solid wound dressing operate under a significant mechanical constraint due to their binding, covalent or other, to the substrate, the chains in direct contact with tissue are bound, typically covalently, not to the solid substrate directly, but to a “buffer” in between layer that has mobility of its own. This layer displays less mobility than the upper one, but still mobile. This layer, in turn, is bound to an underlying additional layer, displaying less mobility than the one above, and so forth, until the most constrained chains are reached, those that are directly bound to the solid substrate. This can be achieved following various strategies such as judiciously changing the composition and/or molecular weight and/or concentration of the RTR polymer. In other embodiments of this invention, thermosets of controllable degrees of crosslinking, varying also in the molecular weight between crosslink junctions. In yet other embodiments of the invention disclosed hereby, thermoplastic and thermoset RTR polymers are combined to generate the desired mobility gradient, so stable attachment of the RT component to the substrate is achieved, on one hand, while the mobility of the RTR chains are maximized at the very interface between them and the tissue.

In an additional embodiment, combinations of the various strategies described above are taught. For example, the combination of layers of crosslinked PEO-PPO-PEO dimethacrylates, which then are exposed to plasma of ammonia, to generate amine groups on the F127 dimethacrylate chains. These amine moieties are then used as anchoring sites for long RTR chains and the process can be repeated several times.

As already stated, in other embodiments, two or more of the embodiments described hereby, are combined, to generate wound dressings with the advantageous features described.

It must to be understood that the examples and embodiments described hereinabove are for the purposes of providing a description of the present invention by way of example and are not to be viewed as limiting the present invention in any way. Various modifications or changes that may be made to that described hereinabove by those of ordinary skill in the art are also contemplated by the present invention and are to be included within the spirit and purview of this application.

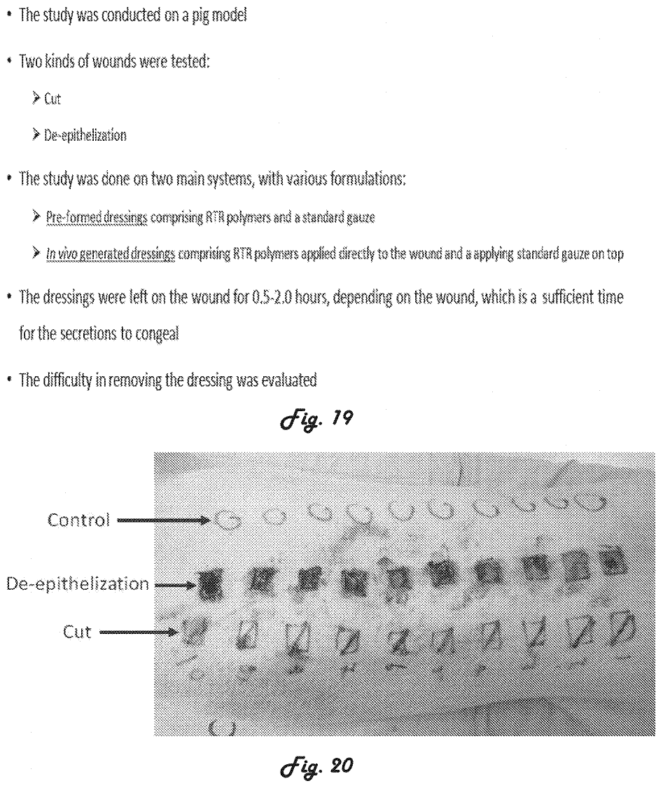

An animal study was conducted using several of the compositions disclosed hereby, as described in FIG. 19. As described there, besides the control, two types of wounds were done on the back of the pig, namely a cut and a more serious de-epithelization.

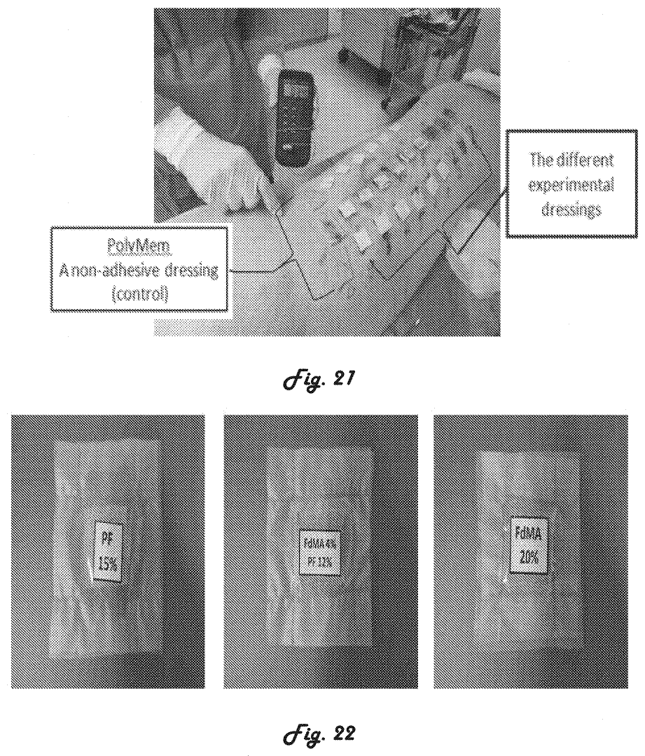

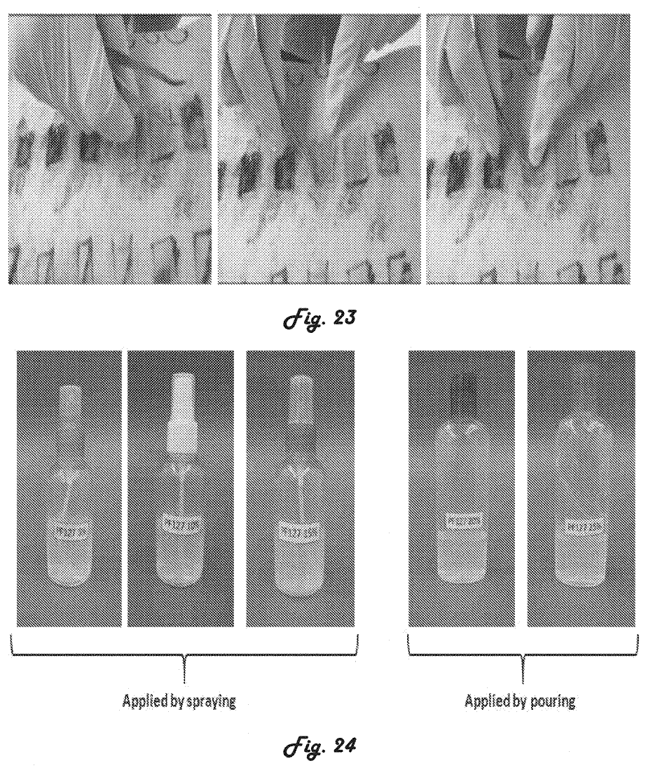

FIGS. 20-21 show two types of wounds and the bare pig skin, as well as the different dressings used, which were also compared with PolyMem, a commercially available dressing that claims to be non-adherent. FIG. 22 shows three of the numerous compositions of pre-formed dressings comprising the RTR component and a dressing, in this case a gauze. FIG. 23 shows pre-formed dressings are shown when being deployed on the back of a pig.

As taught by this invention, also in situ generated dressings comprising only RTR components were developed, and five of them, three that are sprayed on the wound and two that are poured on it, are shown in FIG. 24. In this embodiment, the dressing consists only of a PF polymer, covering different concentrations (from 5% to 25%), and they are engineered so they are deployed spraying them (at lower concentrations) or by pouring then (at higher concentrations) on the wound.

Following the application of the RTR component to the tissue bed, generating a liquefiable interface with it, a dressing of any type, such as, without limitation, a gauze is applied on top of the RTR component. Additionally, the RTR component can be such that it has the required properties to perform as an in situ generated dressing that, in this case will be fully liquefiable, not requiring an additional dressing on top of it.

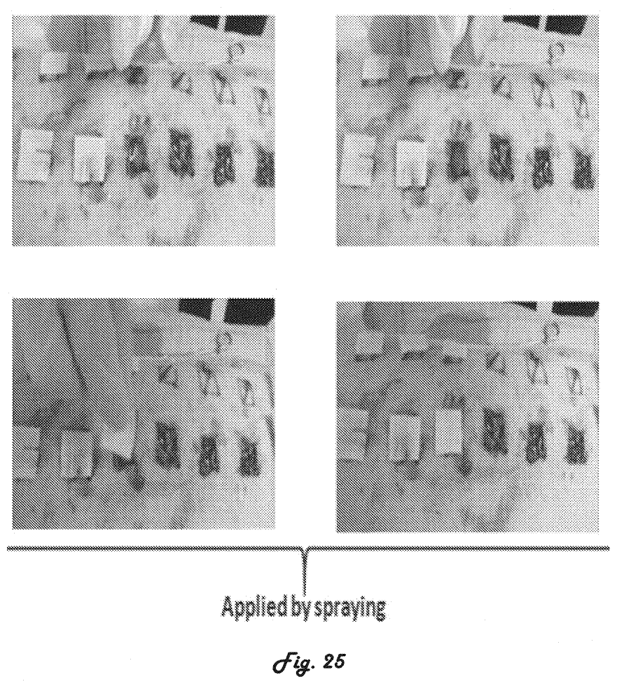

FIG. 25 shows dressings where the RTR component is first sprayed on the wound, followed by the application of a gauze, as just one of the type of dressings that can be used to this effect.

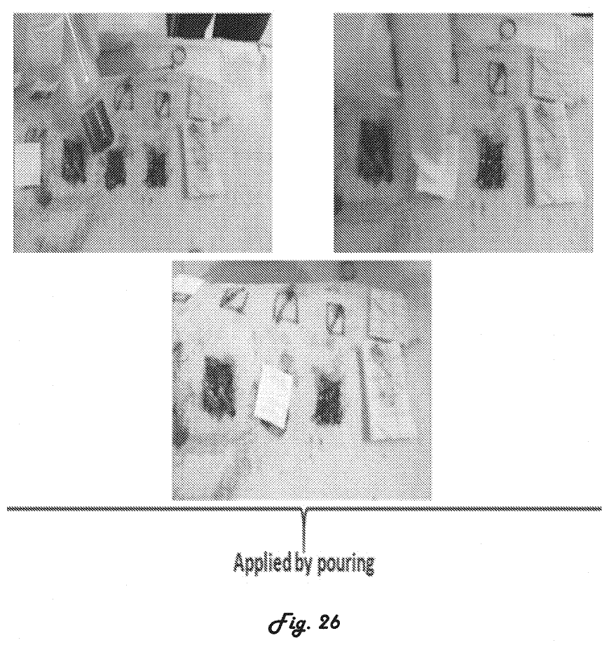

In FIG. 26, dressings where first the RTR component are poured on the wounds on the back of a pig, followed by the application of a dressing, a gauze in this case.

Also in this embodiment, depending on their composition and other parameters, there may be no need for an external dressing to be used on top of the RTR liquefiable component. In these cases, the RTR layer itself is rendered with all the features and properties of a dressing, generating, therefore, a fully liquefiable one.

FIG. 27 shows the wounds when treated with a gauze, with Biatain Ibu, a commercially available dressing that claims to be non-adherent, and the Smart Wound Dressing (SWD).

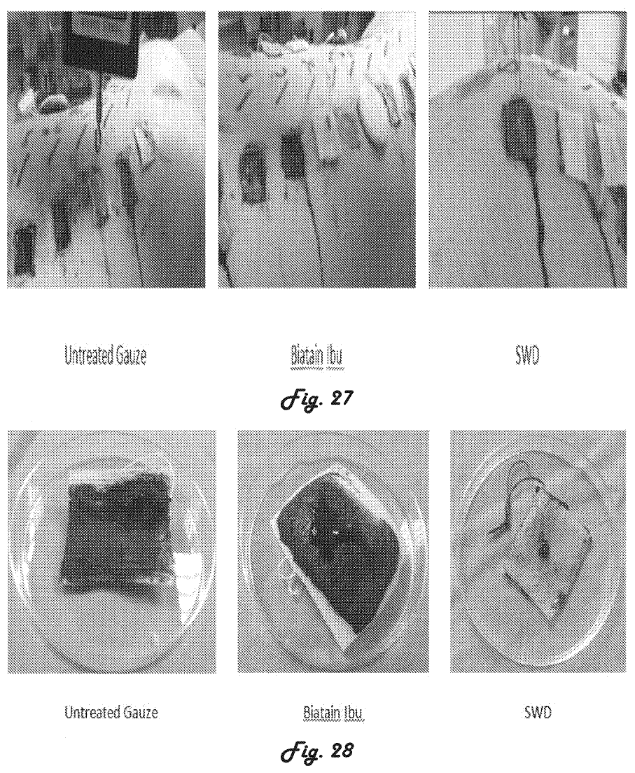

FIG. 28 shows the three dressings, after removal from the wound, compellingly demonstrated the superior performance of the SWD, comprising the RTR component and the liquefiable interface it generates with the tissue bed.

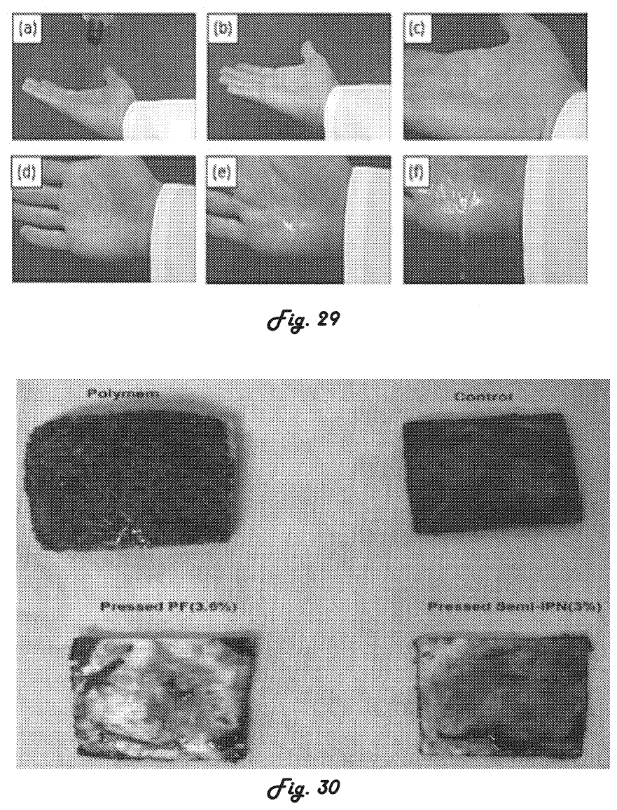

FIG. 29 describes one of the experiments performed whereby an in situ generated, fully liquefiable dressing, consisting only of RTR polymers, were developed. In this embodiment, the RTR formulation is poured on the wound at a suitable temperature, below its relevant thermal sol-gel transition, so it is a solution, the viscosity of which can be tuned. Onc in contact with the wound (in this case, with the skin of the hand), the RTR solution heats up, crossing its thermal transition and gelling on the wound. Subsequently, when required, the dressing is cooled down by various means, in this case using a cooling spray, whereby the gel is liquefied and easily and more importantly, non-injuriously removed from the wound.

In some embodiments, additional components are added to the RTR components. Seeking to improve some of its properties or rendering it with additional capabilities, such as the release of bioactive molecules, such as drugs, among others. One the additives added to the RTR system is a tissue or mucoadhesive component, aiming at optimizing the attachment of the in situ generated dressing to the tissue. This may include the attachment to both the wound as well as the surrounding healthy tissue and each case it may have different objectives.

Polyacrylic acid of various molecular weights, for example 2,000 and 450,000, is one of the additives used. In some embodiments the polyacrylic acid was added to the RTR aqueous solutions and in other instances it was used before the RTR component, as some kind of primer, generating a thin layer in direct contact with the tissue, on one side, and with the RTR dressing on the other side.

The list below presents some of the numerous in situ generated fully liquefiable dressings disclosed hereby.

PF, PF+PAA2000 with Calculated Ratios:

| | |
|---|---|
| PF(13%) + PAA2000(5%) | (18%) |
| :28 | |
| PF(14.5%) + PAA2000(3.5%) | (18%) |
| :20 | |
| PF(14.5%) + PAA2000(6.25%) | (21%) |
| 70:30 | |
| PF(17%) + PAA2000(3.75%) | (21%) |
| :20 | |
| PF(18%) + PAA2000(7%) | (25%) |
| 72:28 | |
| PF(19%) + PAA2000(5%) | (25%) |
| :20 | |
| PF(19.5%) + PAA2000(8%) | (28%) |
| :30 | |
| PF(19%) + PAA2000(8%) | (27%) |
| 80:20 | |

The in situ generated fully liquefiable dressing can be deployed using a variety of techniques, such as, without limitation, spraying, pouring or using a spatula. This will depend on clinical considerations such as the characteristics of the wounded site, among others, as well as the composition, molecular weight and other features of the RTR system. Additionally, other technical issues, such as the quality of the spraying system, for example, play an important role.

In some embodiments the RTR systems consists of a semi-IPN or IPN of special compositions and characteristics. In some embodiments, the IPN and semi-IPN systems disclosed hereby comprise methacrylates of RTR polymers such as F127dMA. In the case of the semi-IPNs, in some embodiments they comprise F127DMA, for example, and different PF polymers consisting of chain extended F127 triblocks, without limitation, using bifunctional molecules of various types, such as diisocyanates, to generate the PF chain extended polymer. Additionally, in the case of both IPN and semi=IPNs systems, the degree of methacrylation of the RTR polymers, meaning, the number of C═C bonds per RTR molecule, is controlled, whereby different RTR systems displaying different molecules, are formed. The list below, describes some of the semi-IPN systems developed.

### PF, PF+FdMA:

PF(10%)

PF(10%)+FdMA(2%)

PF(10%)+FdMA(3%)

PF(10%)+FdMA(4%)

PF(10%)+FdMA(5%)

PF(15%)

PF(15%)+FdMA(2%)

PF(15%)+FdMA(3%)

PF(15%)+FdMA(4%)

PF(15%)+FdMA(5%)

PF(17%)

PF(17%)+FdMA(2%)

PF(17%)+FdMA(3%)

PF(17%)+FdMA(4%)

PF(20%)

PF(20%)+FdMA(2%)

PF(20%)+FdMA(3%)

PF(25%)

PF(25%)+FdMA(2%)

PF(25%)+FdMA(3%)

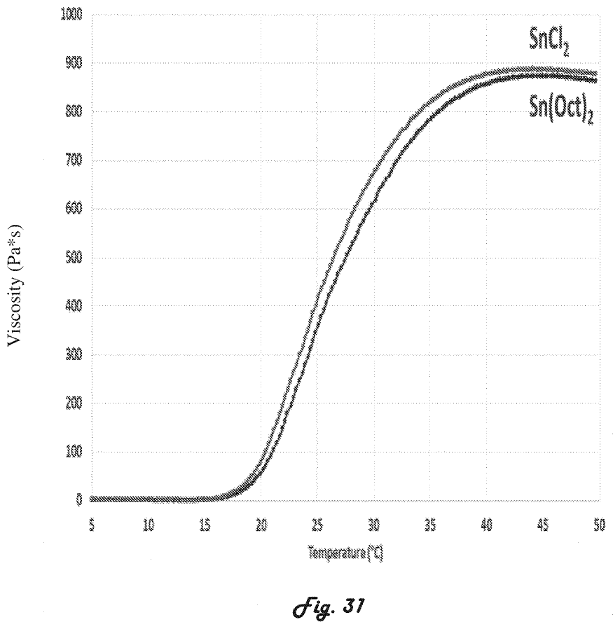

Reference is now made to FIG. 30. Additional in-vivo study presents the removal of a partially liquefiable dressing at the pig model. Polymem commercial wound dressing is compared to the untreated dressing (control) and SWD (with PF or semi-IPN). Pressed fibers covered with semi-IPN or PF, were detached easily with significantly less amount of blood on the dressing, comparing to the control or to the “non-adherent” commercial wound dressing, which were fully absorbed with blood.

For the sake of the development process we will need to produce in situ generated layer on the wound bed that will be able to retain the maximum water amount. Therefore, in some embodiments the RTR systems consists Poly (Ethylene glycol), commercially available Tetronic1307®, Alginate, commercially available F87 or chitosan.

In another embodiment, the wound dressings disclosed hereby will be generated as a thin film composed of PF127 and Poly (Acrylic Acid)/commercially available Tetronic1307/Chitosan and this polymeric patch can also be combined with non-woven fabric.

Reference is now made to FIG. 31. A new synthesis of PF127, based on INCI-COSING approved materials, was conducted. Commercially available Pluronic F127 (molecular weight 12,600) was poured in a three-necked flask and dried. Then, a corresponding amount of HDI and SnCl₂ (CAS # 7772-99-8) (0.64 wt %) were added to the reaction mixture and reacted at 80° C. for 30 minutes under mechanical stiffing (160 RPM) and dry nitrogen atmosphere. The polymer produced was dissolved in chloroform and precipitated in a petroleum ether 40-60 ethyl ether mixture (1:1). Finally, the polymer was washed repeatedly with portions of petroleum ether and dried. Different F-127/HDI ratios resulted in different degrees of polymerization (DP).

Work was also devoted to form short semi-crystalline hydrophobic segments of different length along the chain, able to fine tune the hydrophilicity of the gel and create reversible, physical crosslink domains. This was achieved by reacting the hydroxyl terminal groups of PEO-PPO-PEO triblocks with lactam and lactone rings (e.g. caprolactam and caprolactone, respectively), via a controlled ring opening polymerization reaction. The hydroxy end capped pentablocks initially formed were then chain extended by reacting them with HDI, to produce the high molecular weight RTR polymers. The key drawback of this working concept was that only polymers containing very short hydrophobic segments were water soluble. As a result, those that were able to generate aqueous solutions, were only marginally improved, while those that contained significant hydrophobic domains, prevented the polymer from dissolving in water.

Another strategy that was investigated focused on forming slightly crosslinked thermoset RTR polymers that combine both enhanced stability during service and effective liquefiability during cooling and removal. These polymers were synthesized by reacting F127's terminal OH groups with isocyanate ethyl methacrylate (IEMA) at different ratios, so to generate different degrees of methacrylation. The key limitation of this approach was that, while showing significant potential, but the delicate and time consuming fine tuning work required is beyond the scope of the current project.

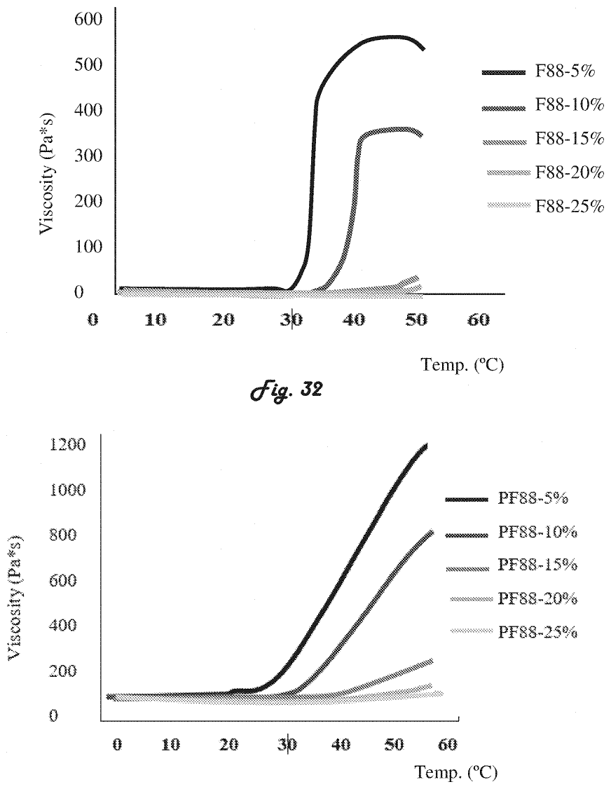

Capitalizing on findings obtained in the lab which demonstrated that triblocks that are more more hydrophilic than F127 (70% wt EO content) produce more cohesive gels, the scope of the project was broadened to include them. Most of the work conducted along this pathway concentrated on the PEO₁₀₂-PPO₃₉-PEO₁₀₂ (F88) triblock (80% wt EO content). Rheometry measurements confirmed that the F88-based RTR-displaying polymers form gels displaying enhanced viscosity, as shown in FIG. 32, namely viscosity versus temperature plots of F88 and PF88.

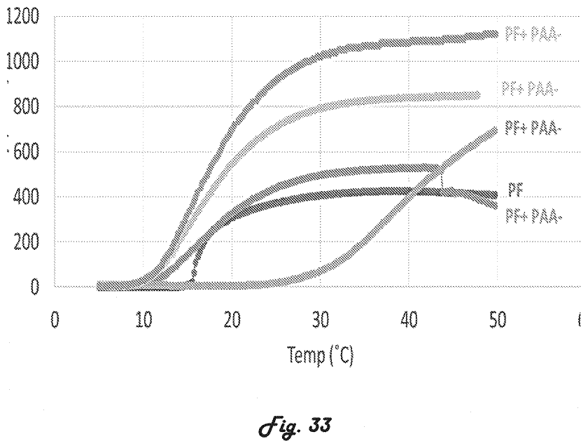

Since these gel were slower to liquefy, when compared with the PF127 polymers, F88 and F127 were co-polymerized, aiming at combining the advantageous features of each of them, while avoiding or minimizing their respective shortcomings. Additionally, much work was allocated to rendering the in situ generated dressing with the required adhesiveness. This was achieved by adding polyacrylic acid (PAAc) chains of various molecular weights, spanning from 2,000 Da to 450,000 Da. FIG. 33 presents the temperature dependent rheological behavior of PF127 25% solutions, containing PAAc chains of different molecular weight. In accordance with theoretical considerations, the longer ther chain, the more effective it is in connecting via hdrogen bonds different PF supramolecular structures. FIG. 33 hence shows viscosity versus temperature plots of PF127 25% solutions, containing PAAc chains of different molecular weight.

The content of the PAAc chains in the systems was optimized, so that the required adhesiveness was achieved with the lowest PAAc content. In the case of PAAc 450,000, the most effective PAAc chain, its optimal content was 0.175%.

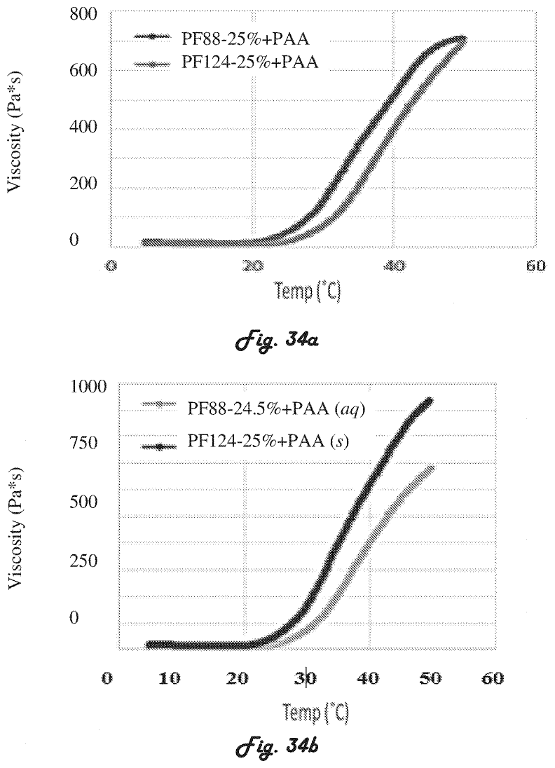

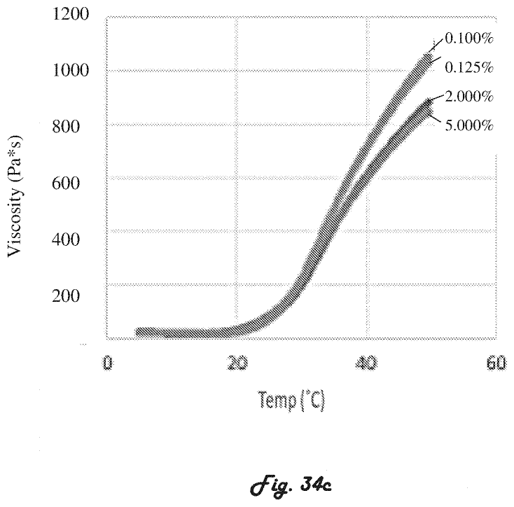

FIGS. 34 and 35 show the rheological measurements of RTR solutions and gels, combining F127, F88 and PAAc 450,000, each of the components contributing specific beneficial functional features to the behavior of the wound dressing. FIG. 34 hence presents viscosity versus temperature curves of F127/F88/PAAc 450,000 systems. FIG. 35 presents viscosity versus temperature curves of five F127/F88/PAAc 450,000 compositions.

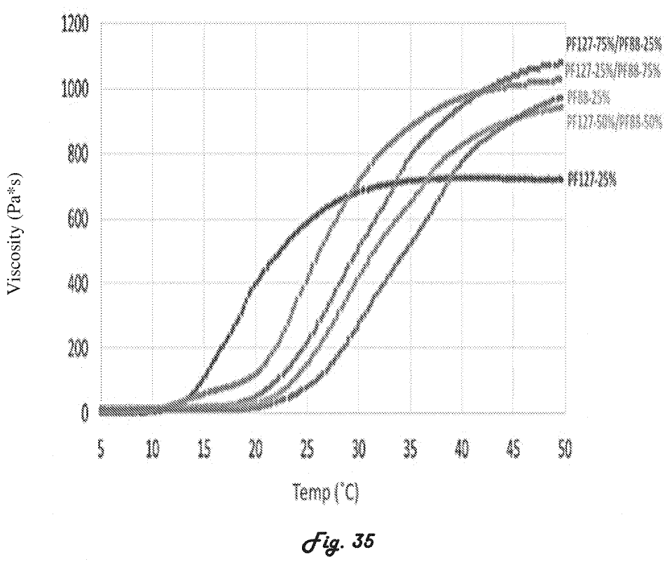

Once the composition of the RTR-displaying dressing was determined, its behavior was studied both in vitro as well as in vivo. The in vitro work was conducted using the chicken skin model, as shown in FIG. 36, following the protocol shown in the figure. The chicken skin in vitro model of a PF88-25%+PAA dressing.

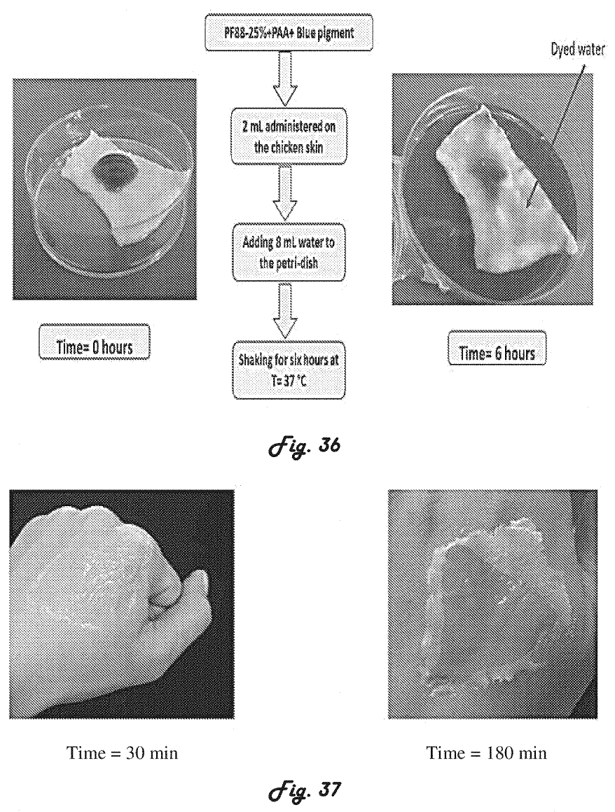

The behavior of the in situ generated dressing was also assessed on healthy living human skin (see FIG. 37) using a PF88-25% dressing without PAAc. A PF88-25% dressing without PAAc on healthy living human skin.

FIG. 38 shows in situ generated dressing on injured living human skin, namely, the dressing a few hours after being deployed on injured living human skin.

The dressing was removed after several hours by liquefying it under cold water and no detrimental effect on the injury was observed.

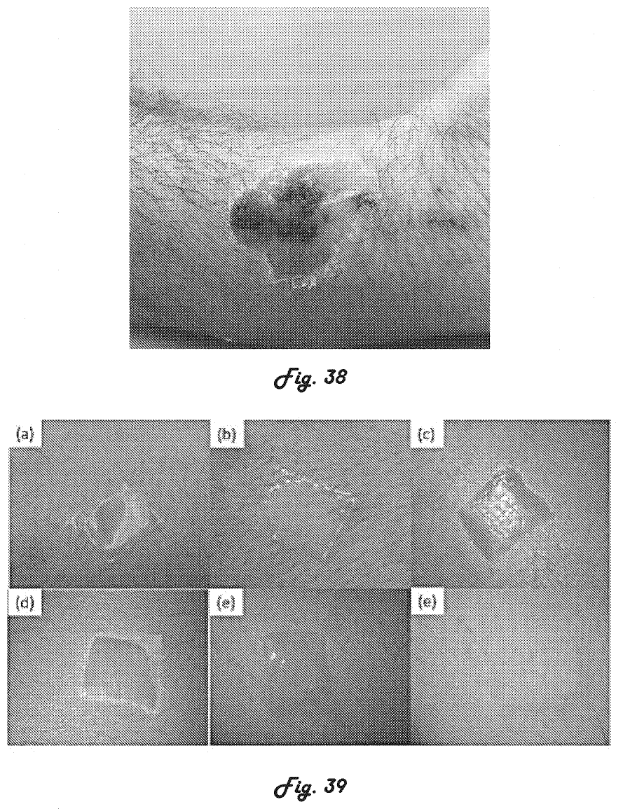

A dry, film version of the PF88-25%+PAA(450k)-0.175% gel was also developed and deployed initially on healthy living human skin, as shown in FIG. 39. The sequence of photos shown, presents the chronology of events that the use of the film entails, from its deployment on moist skin, during its performance over four hour and until its removal using cold water. In FIG. 39, SLB wound dressing is depicted at different stages: (a) Deployment step—wetting; (b) after 1 minute on the skin; (c) after 4 hours on the skin; (d) after 8 hours on the skin; (e) after washing with warm water; (f) after removal by cold water.

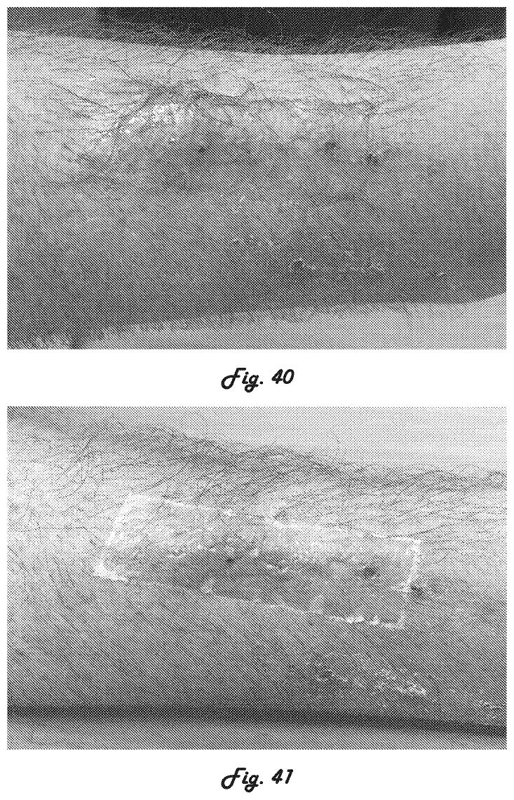

The photos presented in FIGS. 40 and 41 show the dry film PF88-25%+PAA(450k)-0.175% dressing, upon deployment and several hours later, respectively. FIG. 40 hence shows the dry film PF88-25%+PAA(450k)-0.175% dressing upon deployment. FIG. 41 discloses the dry film PF88-25%+PAA(450k)-0.175% dressing several hours after deployment.

The dressing was left on the injury for 72 hours and it exhibited enhanced stability and no harmful effect on the injury could be observed during performance or upon removal of the dressing by liquefaction.

The last experiment conducted was the in vivo study conducted on a pig de-epithelialized skin model at Beilinson Hospital (Israel). FIGS. 42 shows behavior of three different gel dressings, seven minutes and three hours after deployment. Three different gel dressings, seven minutes and three hours after deployment are disclosed.

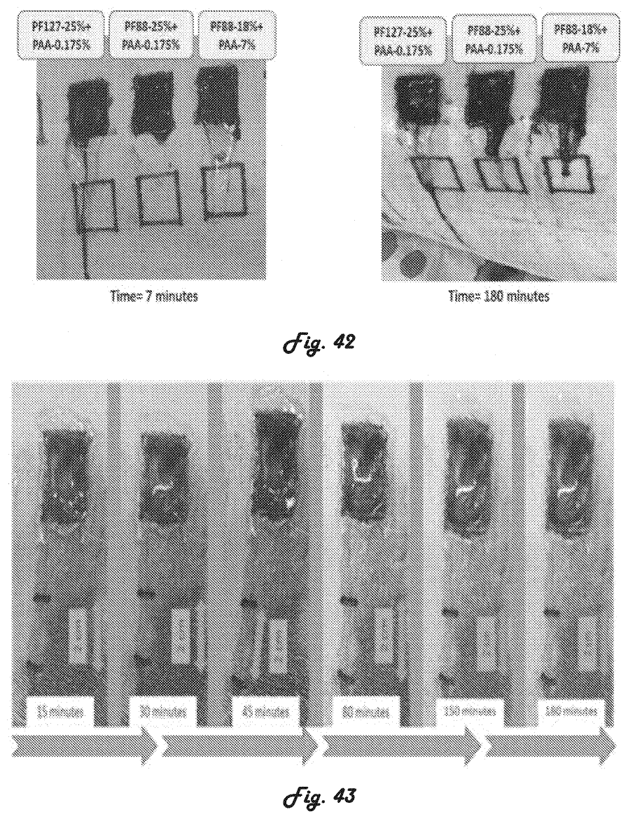

FIG. 43 presents the progression of the treatment over time, on the wound made on the pig's back, showing the stability of the dressing in vivo. In this figure, in vivo progression of the treatment of a PF127 25% dressing over three hours.

Concluding, RTR-displaying polymers were synthesized and analyzed, and two classes of dressings were developed, both sharing the ability to be removed by cooling and liquefaction. The first category of dressings is based on deploying the RTR solution which, on contact with the wound, heats up to physiological temperature and gel, generating a fully conformable dressing. The second approach pursued applied a dry film of the RTR-displaying polymer that attaches effectively to the moist skin, generating the dressing. The performance of both types of dressings was investigated and working prototypes of each were developed and performed very successfully both in vitro as well as in vivo.

### Foam Method

The foam is formed by mixing the PF127 solution in a high velocity propeller. In order to generate a stable foam, the PF concentration in water should be at least 3% s/s. The advantage of this method is that by applying the RTR solution on the fabrics, the foam covers more efficiently the surface and prevents the polymer from penetrating deep inside (see FIG. 16). In addition, at elevated temperature (120° C.) coating the fabric by the foaming method improves the honey test results (see table 1a). Furthermore, no difference detected at the honey test results comparing alternative methods of coating with foam, either by spraying or by spreading (table 1b).

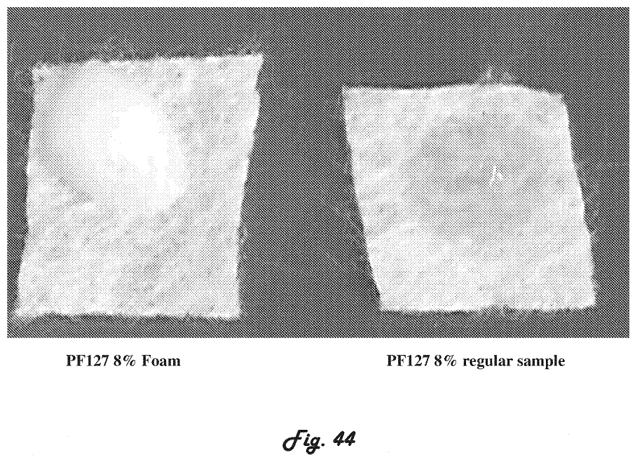

In another embodiment of FIG. 44, the RTR components may be foamed directly on the skin as the protective layer. In FIG. 44, a comparison of fabrics coated by foaming (left) and by regular spreading (right) is depicted.

Table 1 and Table 2—Honey test results of the foaming technique, comparing different temperatures (a) and methods (b).

TABLE 1a

| | | Reducing absorption of honey |
|---|---|---|
| Type | Solid/Solid % | compared to the control (%) |
| Foam 3% at 5° C. | 121 | 28 |
| Foam 3% at 120° C. | 128 | 78 |

TABLE 1b

| | | Reducing absorption of honey |
|---|---|---|
| Type | Solid/Solid % | compared to the control (%) |
| Foam 3% spreading | 106 | 36 |
| Foam 3% spraying | 108 | 37 |

In another embodiment, the RTR components may be welded to another layer, which will maintain its humid nature. The welding may be processed with another RTR polymeric component, with a non-RTR polymeric component and with a fabrics-based component.

It is another embodiment of this invention that any of the components of the compositions disclosed hereby may exhibit shape memory capabilities by incorporation of crystallizable segments, such as PLA, PCL etc. These compositions may also include magnetic nano particles as actuators for the shape memory.

It is another embodiment of this invention that any of the smart wound dressings disclosed hereby, may contain nano-suitcases as drug carriers.

It is another embodiment of this invention where transdermal active molecule delivery systems are disclosed.

### EXAMPLE 1 CBD Active Molecule Delivery

Cannabidiol (CBD) and other plant-based endocannabinoid receptor analogs (ERA) have been examined extensively as compounds of interest in the clinical treatment of pain, immune modulation, and as treatments for neurological disease. This class of phytochemical covers a wide range of compounds originally isolated from the Cannabis sativa plant. CBD and other ERA have chemical structures that interact with a set of chemical receptors in mammalian tissue; most important of these are the CB₁ and CB₂ receptors. Δ⁹-tetrahydrocannabinol (THC) is the most famous ERA and it is the cause of the cannabis “high” neurological effect; this effect is theorized to be largely due to interaction between the molecule and the CB₁ receptor. CBD and other compounds of relevance to this program work either indirectly with the mammalian endocannabinoid system or bind preferentially to the CB₂ receptor. This means that these compounds can have profound curative effects while not causing the altered mental state or disorientation associated with traditional cannabis. The CB₂ receptor has become a target for pharmacological research into pain relief specifically, as cellular signaling associated with positive (agonistic) stimulation of this receptor correlates with immune messaging to reduce inflammation.

The mammalian endocannabinoid system of receptors is distributed throughout all tissues in the body and are most frequently associated with neurological and immune systems, however significant receptor concentration is found directly in the skin. Thus, in addition to inhalation or ingestion of ERA, topical application can result in interaction and a positive clinical response. In the current market, this has resulted in a proliferation of topical salves, lotions, ointments, and other simple delivery vehicles of ERA either as isolates of specific compounds or combinations of hemp extractives. These products are normally oil based to dissolve the ERA and depend on the natural affinity of the ERA molecule for lipids within the skin to transport the molecule into cells. This is completely passive and does not typically result in significant bodily uptake of the molecule. In traditional applications, doses of topical ERA have been extremely high to counteract the apparently low bioavailability of the molecule to provide pain relief; this is financially and pharmacologically inefficient as a treatment. In some applications compounds that improve skin permeability (e.g. dimethylsulfoxide, stearic acid) have been used to improve bioavailability, but these approaches still use the same fundamental vehicle of a lotion or cream that can be easily removed before the molecule of interest is effectively delivered to the target. No company, to our knowledge, has provided a vehicle for delivering ERA to the skin for long periods of time which is safe and effective as a wound dressing.

It is in the scope of the invention wherein the concentration of CBD and/or THC is in the range of 0.1% or less. Alternatively, the range varies from about 0.1% to about 1.5%. Alternatively, the range varies from about 1.5% to about 5%. Alternatively, the range varies from about 5% to about 15%. Alternatively, the range varies from about 15% to about 35%.

The term “about” refers hereinafter to any value being greater than or lower than up to 20% of the defined measure.

Advancements in drug delivery are used to improve ERA delivery beyond a simple topical application into a true transdermal compound delivery system. Some manifestations of this technological advancement have included the generation of specialized emulsions or encapsulations of compounds that have low bioavailability. The specific approaches believed to be most compatible with the proposed product include nano-emulsification and nano-particle delivery vehicles. These approaches offer specific advantages over the current state of topical ERA products: (A) Significantly higher surface area to volume ratio than bulk product, increasing the potential for interaction between the molecule and target tissues—increased bioavailability. (B) Smaller particle sizes, especially those on the scale of proteins (less than 100 nanometer diameter) are more capable of transporting within cellular matrix, thus more available for biological response. (C) Nano-emulsion and encapsulation allows for incompatible chemical types (water soluble and oil soluble for example) to be mixed in stable forms.

It is another embodiment of this invention wherein a wound dressing is disclosed. The wound dressing comprises at least one first measure of an RTR agent; and an active component (AC) integrated within the RTRA.

It is another embodiment of this invention wherein the AC is selected from at least one member of a group consisting of one or more INCI [cosmetics].

It is another embodiment of this invention wherein the cosmetic agent is selected in a non-limiting manner form sunscreen agent, a skin softener, a moisturizing agent, an emollient, and the like.

It is another embodiment of this invention wherein AC is selected from at least one member of a group consisting of one or more nutraceuticals.

It is another embodiment of this invention wherein AC is selected from at least one member of a group consisting of one or more medicaments.

It is another embodiment of this invention wherein AC is selected from at least one member of a group consisting of cannabinoids, CBD, THC, cannabis and extracts thereof, hereinafter cannabinoids.

It is another embodiment of this invention wherein AC is selected from at least one member of a group consisting of household agents.

It is another embodiment of this invention wherein AC is selected from at least one member of a group consisting of agricultural agents.

When the active agent is an agricultural agent, it may be, for example, an herbicide, a pesticide, a fungicide, a rodenticide, a plant nutrient, or a growth hormone or a combination of one or more such agents.

It is another embodiment of this invention wherein AC is selected from at least one member of a group consisting of industrial chemical agents.

It is another embodiment of this invention wherein the wound dressing is utilized in an application selected from a group consisting of vaginal, anal, rectal, proctology, dermal, buccal, growth factors, H acid, trichology, NACS/NSID (anti-inflammatory), ontological preparation (EARS plug), anti-fungal (chlorohexidine), sun screens UV blockers, anti-aging serum, mucosal preparations, veterinary products.

It is another embodiment of this invention wherein the wound dressing comprises RTRs as those selected from a group consisting of natural polymers, N-isopropylacrylamide (NiPAAM) polymers, PEO/PPO-based systems: poly(ethylene oxide)-b-poly(propylene oxide)-b-poly(ethylene oxide) (PEO-PPO-PEO) as well as poly(ethylene glycol) (PEG)-biodegradable polyester copolymers, Poly(organophosphazenes), reversely thermo-reversible hydrogel using water soluble block copolymers of polyethylene oxide and polypropylene oxide available commercially as Pluronic® from BASF (Ludwigshafen, Germany) and generically known as Poloxamers. It is in the scope of the invention wherein the concentration of RTR is in the range of 0.1% or less. Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

It is in the scope of the invention wherein the monomer repeating units are derived from an aliphatic hydroxy carboxylic acid or a related ester, lactone, dimeric ester, carbonate, anhydride, dioxanone, amide, or related monomer, and preferably derived from an aliphatic α-hydroxy carboxylic acid or related ester, such units derived from the following: including, for example, lactic acid, lactide, glycolic acid, glycolide, or a related aliphatic hydroxyl carboxylic acid, ester (lactone), dimeric acid or related compound such as, for example, β-propiolactone, ε-caprolactone, δ-glutarolactone, δ-valerolactone, β-butyrolactone, pivalolactone, α,α-diethylpropiolactone, ethylene carbonate, trimethylene carbonate, γ-butyrolactone, p-dioxanone, 1,4-dioxepan-2-one, 3-methyl-1,4-diox ane-2,5-dione, 3,3 ,-dimethyl-1-4-dioxane-2,5-dione, cyclic esters of α-hydroxybutyric acid, α-hydroxyvaleric acid, α-hydroxyisovaleric acid, α-hydroxycaproic acid, α-hydroxy-α-ethylbutyric acid, α-hydroxyisocaproic acid, α-hydroxy-α-methyl valeric acid, α-hydroxyheptanoic acid, α-hydroxystearic acid, α-hydroxylignoceric acid, salicylic acid and mixtures thereof. It is in the scope of the invention wherein the concentration of monomer is in the range of 0.1% or less. Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

It is another embodiment of this invention wherein the wound dressing is selected from a group consisting of Wound dressing, Patch, Foam, Film, Gel, Hydrocolloid, Hydrogel, Alginate, Collagen, Transparent, Cloth, Gauze, Gauze Sponge, Gauze Bandage Roll, Non Adherent Pads, Non Adherent Wet Dressings, Alcohol Preps, Antimicrobials, Cadexomer Iodine, Composites, Gelling Fibers, Honey (medical grade), Negative Pressure Wound Therapy (NPWT), Petrolatum and Oil Emulsions, Silicone Sheets, Specialty Absorbents/Super Absorbents, Wound Filler, Contact Layers, Transparent Film, Wound Filler, Beads, creams, pillows, gels, ointments, pastes, pads, powders, strands, Topical Antifungals: Antifungal creams, powders, liquids, or sprays are used to treat fungal infections of the skin. They include clotrimazole, econazole, ketoconazole, miconazole, tioconazole, terbinafine, and amorolfine, Topical Antibiotics: Antibiotic creams and/or ointments, such as bacitracin, triple antibiotic ointment (polymyxin B, neomycin, bacitracin), gentamicin, mupirocin, and erythromycin, are used to treat skin infections, Topical Corticosteroids: alclometasone dipropionate (Aclovate), desonide (Desowen, Verdeso), and hydrocortisone (Hytone). betamethasone valerate (Luxiq), clocortolone pivalate (Cloderm), fluocinolone acetonide (Synalar), flurandrenolide (Cordran), fluocinonide (Lidex), fluticasone propionate (Cutivate), hydrocortisone butyrate (Locoid), hydrocortisone valerate (Westcort), mometasone furoate (Elocon), and prednicarbate (Dermatop). amcinonide (Cylocort), desoximetasone (Topicort, Topicort LP), halcinonide (Halog), clotrimazole-betamethasone diproprionate (Lotrisone), and triamcinolone acetonide (Kenalog). betamethasone dipropionate (Diprolene), clobetasol propionate (Clobex, Temovate, Olux), diflorasone diacetate, fluocinonide (Vanos), tacrolimus (Protopic), and halobetasol propionate (Ultravate). Topical Antiseptics: Acetic Acid, Cadexomer Iodine, Chlorhexidine Gluconate, Dialkycarbmoyl Chloride (DACC), Hexachlorophne, Hydrogen Peroxide, Iodine Compounds/Tincture, Sodium Hypochlorite. Topical Antimicrobials: Medical Grade Honey. Silver Dressings: Silver Ion, Silver Nitrate, Silver Sulfadiazine, Gentian Violet, Methelyne Blue Non-adhesive dressings, made of natural material, e.g. cellulose-, protein-, collagen-based, synthetic polymer based, Wound bandages, poly(N-vinyl lactam)-chitosan gels, polyvinylpyrrolidone (PVP) and any combination thereof. It is in the scope of the invention wherein the concentration of active agent is in 0.01% (wt/wt) or less. Alternatively, the range varies from about 0.02% (wt/wt) to about 0.1% (wt/wt). Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

It is another embodiment of this invention wherein the cannabinoid is selected from one or more members of a group consisting of THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), CBT (cannabicitran); Endocannabinoid ligands such as Arachidonoylethanolamine (Anandamide or AEA), 2-Arachidonoylglycerol (2-AG), 2-Arachidonyl glyceryl ether (noladin ether), N-Arachidonoyl dopamine (NADA), Virodhamine (OAE), Lysophosphatidylinositol (LPI); Synthetic cannabinoids, such as Dronabinol (Marinol), Nabilone (Cesamet, Canemes), Rimonabant (SR141716), JWH-018, JWH-073, CP-55940, Dimethylheptylpyran, HU-210, HU-331, SR144528, WIN 55,212-2, JWH-133, Levonantradol (Nantrodolum), AM-2201, Cannabicyclohezanol (CP47,497-C8), JWH-108, JWH-073, JWH-122, JWH-200. It is in the scope of the invention wherein the concentration of cannabinoid is in 0.01% (wt/wt) or less. Alternatively, the range varies from about 0.02% (wt/wt) to about 0.1% (wt/wt). Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

It is another embodiment of this invention wherein cosmetic use is selected from one or more members of a group consisting of skin's protective articles, clothing, prosthesis, heat wraps, pads, packs, cold wraps, protective face masks, ornamental articles or eye wear, functional articles being cosmetic or pharmaceutical delivery articles, attachment to the skin, particularly for the adhesion of protective articles such as genital-, knee- or elbow-protectors or bandages; clothing such as bras, surgical gowns, or parts of garments during fitting at a tailor; nasal plasters; prosthesis such as breast replacements or wigs; heat wraps, pads, and/or packs, e.g. for topical relief of pain or simply to provide warmth; cold wraps e.g. to provide pain relieve from bruises and to reduce swelling; protective face masks; ornamental articles such as guises, tattoos; flexible goggles or other eye wear. Further, the combination of the present invention can be also used for application of functional articles to the skin, particularly for the adhesion of functional articles or the improvement of the function of such articles. Functional articles in this context are cosmetic or pharmaceutical delivery articles which provide a substance to the skin such as skin treatment substances, creams, lotions, hormones, vitamins, deodorants, or drugs; alternatively, cosmetic or pharmaceutical delivery articles can also provide a substance to emanate away from the skin such as insecticides, inhalation drugs, or perfumes.

It is another embodiment of this invention wherein a cosmetics active ingredient is selected from one or more members of a group consisting of essential oils, moisture retention agents, skin-beautifying agents, sun screen, antiperspirants, vitamins, amino acids, anti-acne agents, antiseptics or antibacterial agents, zinc salts, tooth whitening agents, depilatory agents, fragrance oils, insect repellants, antioxidants, chelating agents, refrigerants, anti-inflammatory agents, salts, colorants, particulate fillers, baby shampoos, lotions, and creams; bath preparations, such as bath oils, tablet and salts, bubble baths, bath fragrances and bath capsules; eye makeup preparations, such as eyebrow pencil, eyeliner, eye shadow, eye lotion, eye makeup remover and mascara; fragrance preparations, such as colognes and toilet waters, powders and sachets; noncoloring hair preparations, such as hair conditioner, hair spray, hair straighteners, permanent waves, rinses shampoos, tonics, dressings and other grooming aids; color cosmetics ; hair coloring preparations such as hair dye, hair tints, hair shampoos, hair color sprays, hair lighteners and hair bleaches; makeup preparations such as foundations, leg and body paints, lipstick, makeup bases, rouges and makeup fixatives; oral hygiene products such as dentifrices and mouthwashes; personal cleanliness, such as bath soaps and detergents, deodorants, douches and feminine hygiene product; shaving preparations such as aftershave lotion, beard softeners, shaving soap and pre-shave lotions; skin care preparations such as cleansing preparations, skin antiseptics, depilatories, face and neck cleansers, body and hand cleansers, moisturizers, skin fresheners; and suntan preparations such as suntan creams, gels and lotions, indoor tanning preparations. Skin-care creams, lipsticks, eye and facial makeup, towelettes, and colored contact lenses; to the body: antiperspirant/deodorants, including sprays, sticks and roll-on products, lotions, powders, perfumes, baby products, bath oils, bubble baths, bath salts, and body butters; to the hands/nails: fingernail and toe nail polish, and hand sanitizer; to the hair: permanent chemicals, hair colors, hair sprays, and gels. Lipstick, lip gloss, lip liner, lip plumper, lip balm, lip stain, lip conditioner, lip primer, lip boosters, and lip butters. Concealer, Foundation, Face powder, Rouge, blush or blusher, Highlight, Bronzer, Mascara, Eye shadow, Setting spray, False eyelashes, Exfoliants, Moisturizers, Brushes, Mineral makeup, face and neck preparations, insect repellent, styling gels, hair dyes, toners, shaving products, toners, nail polish remover, body and hand preparations, cuticle softeners, aftershaves, non-woven applications for personal care, cucumber slices, scrubbing cleansers, astringents, nail conditioners, hair care pump sprays and other non-aerosol sprays, hair-frizz-control gels, hair leave-in conditioners, hair pomades, hair de-tangling products, hair fixatives, hair bleach products, skin lotions, pre-shaves and pre-electric shaves, anhydrous creams and lotions, oil/water, water/oil, multiple and macro and micro emulsions, water-resistant creams and lotions, anti-acne preparations, mouth-washes, massage oils, toothpastes, clear gels and sticks, ointment bases, topical wound-healing products, aerosol talcs, barrier sprays, vitamin and anti-aging preparations, herbal-extract preparations, bath salts, bath and body milks, hair styling aids, hair-, eye-, nail- and skin-soft solid applications, controlled-release personal care products, hair conditioning mists, skin care moisturizing mists, skin wipes, pore skin wipes, pore cleaners, blemish reducers, skin exfoliators, skin desquamation enhancers, skin towelettes and cloths, depilatory preparations, personal care lubricants, nail coloring preparations, sunscreens, cosmetics, hair care products, skin care products, toothpastes, drug delivery systems for topical application of medicinal compositions that are to be applied to the skin and combinations comprises at least one of the foregoing applications, Body Lotion, Fragrance, Body Wash and Shower Gel/Foam, Hand and Foot and Nail, Treatments, Antiperspirant and Deodorant, Soap, Hair Removal and Shaving, Sun Care, Self-Tanner, Body Scrubs, Bath Salt, Oil, Fizzies and Bubbles, Home Aromatherapy, Nail Tools, Cellulite Treatment, Scar and Stretch Mark Reducers, Body Powder, Bath Brushes, Sponges, Professional Services, Intimate Care, Cleanser, Anti-Aging, Exfoliators, Scrubs, Eye Treatments, Blemish and Acne Control, Masks, Peels, Makeup Remover, Toners, Astringents, Oral Care, Night Cream, Sun Protection, Toothpaste and Breath Freshener, Lip Plumper, Microderm—abrasion, Lightening Cream, Dermatology Treatments & Peels, Ingestibles and Supplements, Lip Exfoliant, Shampoo, Conditioner, Styling Cream, Mousse and Gel, Hair and Scalp Treatments, Hairspray and Finishing Spray, Hair Color, Shine Serum and Gloss, Pomade, Wax and Finishing Cream, Blow Dryers, Brushes and Combs, Hair Accessories, Curling Irons, Rollers, Curlers, Hair Loss, Lip Balm, Primer and Shine Control, Blush, Nail Polish, Kits/Sets, Brow Enhancers, Lip Stain, Lip Liner, False Lashes, Cases, Bags, Totes, PPG-3 myristylether, PEG-16 cetyl/oleyl/stearyl/lanolin alcohol ether, PEG/PPG-4/2 propylheptyl ether, PEG/PPG-6/2 propylheptyl ether, PPG-10 cetyl ether, PPG-20 cetyl ether, PPG-2 lanolin alcohol ether, PPG-5 lanolin alcohol ether, PPG-4 myristyl ether, PPG-11 stearyl ether and PPG-15 stearyl ether and any combination or ingredient thereof. It is in the scope of the invention wherein the concentration of cosmetic active agent(s) is 0.01% (wt/wt) or less. Alternatively, the range varies from about 0.02% (wt/wt) to about 0.1% (wt/wt). Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

It is another embodiment of this invention wherein a nutraceutical is one or more members of a group consisting of vegetable oil selected from the group consisting of wheat, rice, rice bran, corn, millet, sorghum, rye, oats, barley, and combinations thereof, resveratrol and/or its derivatives, cyclodextrin, an adsorbent, an adequate vehicle, trans-3,5-dimethoxy-4′-hydroxy-stilbene, trans-3,5,4″-trimethoxy-stilbene, trans-3,5-hydroxy-4′-methoxy-stilbene, trans-3,5-diacetyl-4′-hydroxy-stilbene, trans-3,5,4″-triacetyl-stilbene and trans-3,5-hydroxy-4′-acetyl-stilbene, and combinations thereof, α-cyclodextrin (α-CD), β-cyclodextrin (β-CD), γ-cyclodextrin (γ-CD), hydroxyethyl-β-CD, hydroxypropyl-β-CD, sulfobutylether-β-CD, methyl-β-CD, dimethyl-β-CD, random dimethylated-β-CD, random methylated-β-CD, carboxymethyl-β-CD, carboxymethyl ethyl-β-CD, diethyl-β-CD, tri-O-methyl-β-CD, tri-β-ethyl-β-CD, tri-O-butyryl-β-CD, tri-O-valeryl-β-CD, di-O-hexanoyl-β-CD, glucosyl-(3-CD, maltosyl-β-CD, and 2-hydroxy-3-trimethyl-ammoniopropyl-β-CD, and combinations thereof, the resveratrol and/or its derivatives are selected from the group consisting of trans-3,5-dimethoxy-4′-hydroxy-stilbene, trans-3,5,4″-trimethoxy-stilbene, trans-3,5-hydroxy-4′-methoxy-stilbene, trans-3,5-diacetyl-4′-hydroxy-stilbene, trans-3,5,4″-triacetyl-stilbene and trans-3,5-hydroxy-4′-acetyl-stilbene, and combinations thereof, an activated fatty acid component selected from the group consisting of nitro-linoleic acid, nitro-α-linoleic acid, nitro-γ-linoleic acid, and combinations thereof; a first non-activated fatty acid component selected from the group consisting of linoleic acid, α-linoleic acid, γ-linoleic acid, and combinations thereof; a second non-activated fatty acid component selected from the group consisting of eicosapentaenoic acid (EPA), docosahexaenoic acid (DHA), and combinations thereof; fatty acid component, vitamin A, vitamin B, vitamin B-1, vitamin B-2, vitamin B-6, vitamin B-12, vitamin C, vitamin D, vitamin D3, vitamin E, selenium, β-carotene, gingko biloba, goldenseal, valerian, ginseng, echinacea, grape seed extract, ephedra, yucca concentrate, green tea extract, rice bran extract, wheat germ, wheat germ extract, beeswax, red yeast rice extract, stevia leaf extract, flaxseed oil, borage seed oil, coenzyme Q10, glucosamine derivatives, methylsulfonylmethane, pantothenic acid, biotin, thiamin, riboflavin, niacin, folic acid, palmitic acid, and combinations thereof. Goldenseal, valerian, ginseng, echinacea, and combinations thereof, Actisaf Sc47 Live Yeast, Bio Available Trace Minerals, Biotin, Calcium, Chelated Minerals, Chondroitin, Collagen, Copper, Oligosaccharides, Manganese, Curcumin, Digestible Milk Proteins, Galactolipid, Glucosamine, Glutamic Acid, Omega 3, Hemp Oil Derived Omega 3 Fatty Acids, Magnesium, Methylsulfonylmethane, Oat Bran, Oatinol, Phospholipids, Prebiotics, Chloride, Sodium, Zinc, and any combination, mixture or derivative thereof. It is in the scope of the invention wherein the concentration of the oil is 0.01% (wt/wt) or less. Alternatively, the range varies from about 0.02% (wt/wt) to about 0.1% (wt/wt). Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

It is another embodiment of this invention wherein a medicament is one or more members of a group consisting of agents for treating cardiovascular conditions such as chlorothiazide (diuretic), propranolol (antihypertensive), hydralazine (peripheral vasodilator), isosorbide or nitroglycerin (coronary vasodilators), metoprolol (beta blocker), procainamide (antiarrythmic), clofibrate (cholesterol reducer) or coumadin (anticoagulant); agents for treating internal conditions such as conjugated estrogen (hormone), tolbutamide (antidiabetic), levothyroxine (thyroid conditions), propantheline (antispasmodic), cimetidine (antacid), phenyl propanolamine (antiobesity), atropine or diphenoxalate (antidiarrheal agents), docusate (laxative), or prochlorperazine (antinauseant); agents for treating mental health conditions such as haloperidol or chlorpromazine (tranquilizers), doxepin (psychostimulant), phenytoin anticonvulsant), levo dopa (antiparkinism), benzodiazepine (antianxiety) or phenobarbital (sedative); anti-inflammatory agents such as fluorometholone, acetaminophen, phenacetin, aspirin, hydrocortisone, or predisone; anti-histamines such as diphenhydramine hydrochloride or dexchlorpheniramine maleate; antibiotics such as sulfanilamide, sulfamethizole, tetracycline hydrochloride, penicillin and its derivatives, cephalosporin derivatives or erythromycin; chemotherapeutic agents such as sulfathiazole, doxorubicin, cisplatin or nitrofurazone; topical anesthetics such as benzocaine; cardiac tonics such as digitalis or digoxin; antitussives and expectorants such as codeine phosphate, dextromethorphan or isoproterenol hydrochloride; oral antiseptics such as chlor hexidine hydrochloride or hexylresorcinol; enzymes such as lysozyme hydrochloride or dextronase; birth control agents such as estrogen; opthalmic treating agents such as timolol or gentamycin, and the like. In addition, medicinal treating agents may also include whole proteins such as the VP3 capsid protein (also known as the VP Thr and VP1 capsid proteins in other nomenclature systems) of foot-and-mouth disease virus described in U.S. Pat. No. 4,140,763 as being useful as the active ingredient in a vaccine against foot-and-mouth disease, insulin or interferon; polypeptide treating agents such as endorphins, human growth hormone, or bovine growth hormone, or still lower molecular weight polypeptides or conjugates of those polypeptides linked protein carriers as are described in Sutcliffe et al., Science, 219, 660-666 (1983); drugs that act upon the central nervous system; drugs affecting renal function; drugs affecting cardiovascular function; drugs affecting gastrointestinal function; drugs for treatment of helminthiasis; antimicrobial agents such as silver, silver compounds, and/or chlorhexidine; nutrients; hormones; steroids; and drugs for treatment of dermatoses; non-steroidal anti-inflammatory drugs such as salicylates e.g., acetylsalicylic acid; propionic acid derivatives e.g., (RS)-2-(4-(2-Methylpropyl)phenyl)propanoic acid (ibuprofen); acetic acid derivatives e.g., 2-{1-[(4-Chlorophenyl)carbonyl]-5-methoxy-2-methyl-1 H-indo1-3-yl}acetic acid (indomethacin), enolic acid derivatives; anthranilic acid derivatives, COX-2 inhibitors e.g., N-(4-hydroxyphenyl)ethanamide N-(4-hydroxyphenyl)acetamide (acetaminophen), and sulfonanilides; local anesthetics such local anesthetics containing an ester group e.g., ethyl 4-aminobenzoate (benzocaine); local anesthetics containing an amide group e.g., 2-(diethylamino)-N-(2,6-dimethylphenyl)acetamide (lidocaine); and naturally derived local anesthetics e.g., (1 ,2SR,5R)-2-lsopropyl-5-methylcyclohexanol (menthol); antibacterial substances such as beta-lactam antibiotics, such as cefoxitin, n-formamidoyl-thienamycin and other thienamycin derivatives, tetracyclines, chloramphenicol, neomycin, carbenicillin, colistin, penicillin G, polymyxin B, vancomycin, cefazolin, cephaloridine, chibrorifamycin, gramicidin, bacitracin, sulfonamides; aminoglycoside antibiotics such as gentamycin, kanamycin, amikacin, sisomicin and tobramycin; nalidixic acid and analogs such as norfloxacin and the antimicrobial combination of flucalanine/pentizidone; nitrofurazones, and the like; antihistaminics and decongestants such as pyrilamine, chlorpheniramine, tetrahydrazoline, antazonline, and the like.

Anti-inflammatories such as cortisone, hydrocortisone, hydrocortisone acetate, betamethasone, dexamethasone, dexamethasone sodium phosphate, prednisone, methylpredinisolone, medrysone, fluorometholone, fluocortolone, prednisolone, prednisolone sodium phosphate, triamcinolone, indomethacin, sulindac, its salts and its corresponding sulfide, and the like; miotics and anticholinergics such as echothiophate, pilocarpine, physostigmine salicylate, diisopropylfluorophosphate, epinephrine, dipivolyl epinephraine, neostigmine, echothiophate iodide, demecarium bromide, carbachol, methacholine, bethanechol, and the like; mydriatics such as atropine, homatropine, scopolamine, hydroxyamphetamine, ephedrine, cocaine, tropicamide, phenylephrine, cyclopentolate, oxyphenonium, eucatropine, and the like; and other drugs used in the treatment of eye conditions or diseases; antiglaucoma drugs, for example, betaxalol, pilocarpine, timolol, especially as the maleate salt and R-timolol and a combination of timolol or R-timolol with pilocarpine. Also included are epinephrine and epinephrine complex or prodrugs such as the bitartrate, borate, hydrochloride and dipivefrin derivatives and hyperosmotic agents such as glycerol, mannitol and urea; antiparasitic compounds and/or anti-protozoal compounds such as ivermectin; pyrimethamine, trisulfapyrimidine, clindamycin and corticosteroid preparations; antiviral effective compounds such as acyclovir, 5-iodo-2′-deoxyuridine (IDU), adenosine arabinoside (Ara-A), trifluorothymidine, and interferon and interferon inducing agents.

Carbonic anhydrase inhibitors such as acetazolamide, dichlorphenamide, 2-(p-hydroxyphenyl) thio-5-thiophenesulfonamide, 6-hydroxy-2-benzothiazolesulfonamide and 6-pivaloyloxy-2-benzothiazolesulfonamide; anti-fungal agents such as clotrimzole, fluconazole, flucytosine, itraconazole, ketoconazole, miconazole, ciclopirox, econazole, nystatin, oxiconazole, terbinafine Hydrochloride, tioconazole, butoconazle, terconazole, miconazole nitrate, metronidazole, isoconazole nitrate, and tolnaftate; anesthetic agents such as etidocaine cocaine, henoxinate, dibucaine hydrochloride, dyclonine hydrochloride, naepaine, phenacaine hydrochloride, piperocaine, proparacaine hydrochloride, tetracaine hydrochloride, hexylcaine, bupivacaine, lidocaine, mepivacaine and prilocaine; ophthalmic diagnostic agents such as: (a) Those used to examine the retina and chloride-sodium fluorescein; (b) Those used to examine the conjunctive, cornea and lacrimal apparatus such as fluorescein and rose bengal; and (c) Those used to examine abnormal pupillary responses such as methacholine, cocaine, adrenaline, atropine, hydroxyamphetamine and pilocarpine; ophthalmic agents used as adjuncts in surgery such as alphachymotrypsin and hyaluronidase; chelating agents such as ethylenediamine tetraacetate (EDTA) and deferoxamine; immunosuppressive agents and anti-metabolites such as methotrexate, cyclophosphamide, 6-mercaptopurine, and azathioprine; peptides and proteins such as atrial natriuretic factor, calcitonin-gene related factor, lutinizing hormone, releasing hormone, neurotensin, vasoactive intestinal peptide, vasopressin, cyclosporine, Botulinum toxin, interferon, substance P enkephalins, epidermal growth factor, eyederived growth factor, fibronectin, insulin-like growth factor and mesodermal growth factor; acne treatment agents, such as salicylic acid and its derivatives, sulfur, lactic acid, glycolic, pyruvic acid, azelaic acid, benzoyl peroxide, urea, resorcinol and N-acetylcysteine, and retinoids, such as retinoic acid, and its derivatives, and the like; lubricating agents such as sodium hyaluronate or polyvinyl alcohol; and Combinations of the above such as antibiotic/anti-inflammatory as in neomycin sulfate-dexamethasone sodium phosphate, concomittant anti-glaucoma therapy such as timolol maleate-aceclidine nonionic surfactants include fatty acid esters of polyols, for-instance sorbitol or glyceryl mono-, di-, tri- or sesqui-oleates or stearates, glyceryl or polyethylene glycol laurates; fatty acid esters of polyethylene glycol (polyethylene glycol monostearate or monolaurate); polyoxyethylenated fatty acid esters (stearate or oleate) of sorbitol; polyoxyethylenated alkyl (lauryl, cetyl, stearyl or octyl) ethers. Examples of anionic surfactants include carboxylates (sodium 2-(2-hydroxyalkyloxy) acetate)), amino acid derivatives (N-acylglutamates, N-acylglycinates or acylsarcosinates), alkyl sulfates, alkyl ether sulfates and oxyethylenated derivatives thereof, sulfonates, isethionates and N-acylisethionates, taurates and N-acyl N-methyltaurates, sulfosuccinates, alkylsulfoacetates, phosphates and alkyl phosphates, polypeptides, anionic derivatives of alkyl polyglycoside (acyl-D-galactoside uronate), and fatty acid soaps, and mixtures thereof. Examples of amphoteric and zwitterionic include betaines, N-alkylamidobetaines and derivatives thereof, glycine derivatives, sultaines, alkyl polyaminocarboxylates and alkylamphoacetates, and mixtures thereof; adrenergic agents such as ephedrine, desoxyephedrine, phenylephrine, epinephrine and the like, cholinergic agents such as physostigmine, neostigmine and the like, antispasmodic agents such as atropine, methantheline, papaverine and the like, curariform agents such as chlorisondamine and the like, tranquilizers and muscle relaxants such as fluphenazine, chlorpromazine, triflupromazine, mephenesin, meprobamate and the like, antidepressants like amitriptyline, nortriptyline, and the like, antihistamines such as diphenhydramine, dimenhydrinate, tripelennamine, perphenazine, chlorprophenazine, chlorprophenpyridamine and the like, hypotensive agents such as rauwolfia, reserpine and the like, cardioactive agents such as bendroflumethiazide, flumethiazide, chlorothiazide, aminotrate, propranolol, nadolol, metoprolol, atenolol, procainamide and the like, angiotensin converting enzyme inhibitors such as captopril and enalapril, bronchodialators such as theophylline, steroids such as testosterone, prednisolone, and the like, antibacterial agents, e.g., sulfonamides such as sulfadiazine, sulfamerazine, sulfamethazine, sulfisoxazole and the like, antimalarials such as chloroquine and the like, antibiotics such as the tetracyclines, nystatin, erythromycin, streptomycin, cephradine and other cephalosporins, penicillin, semisynthetic penicillins, griseofulvin and the like, sedatives such as chloral hydrate, phenobarbital and other barbiturates, glutethimide, antitubercular agents such as isoniazid and the like, analgesics such as aspirin, acetominophen, propoxyphene, meperidine, ibuprofen, and the like, etc. These substances are frequently employed either as the free compound or in a salt form, e.g., acid addition salts, basic salts like alkali metal salts, etc. Other therapeutic agents having the same or different physiological activity can also be employed in pharmaceutical preparations within the scope of the present invention. It is in the scope of the invention wherein the concentration of the medicament is 0.0001% (wt/wt) or less. Alternatively, the range varies from about 0.0002% (wt/wt) to about 0.001% (wt/wt). Alternatively, the range varies from about 0.001% (wt/wt) to about 0.01% (wt/wt). Alternatively, the range varies from about 0.02% (wt/wt) to about 0.1% (wt/wt). Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

It is another embodiment of this invention wherein a household agents is one or more members of a group consisting of Soap or detergent, Ammonia solution, Calcium hypochlorite (powdered bleach), Citric acid, Sodium hypochlorite (liquid bleach), Sodium hydroxide (lye), Acetic acid (vinegar), Isopropyl alcohol or rubbing alcohol, Borax, Sodium bicarbonate (baking soda(, Tetrachloroethylene (dry cleaning), Carbon dioxide, Chromic acid, Trisodium phosphate, Saltwater soap, Sodium percarbonate, Sodium perborate, Acetone, Amyl nitrite and other nitrites, Xylene, Freon (e.g. dichlorodifluoromethane). Abrasive Cleaners: Powders, Liquids, Scouring Pads, Non-abrasive Cleaners: Powders, Liquids, and Sprays. Kitchen, Bathroom, Glass and Metal Cleaners: Bleaches (chlorine bleach, a cleaner, a fish tank preservative or the like), Disinfectants and Disinfectant Cleaners, Drain Openers, Glass Cleaners, Glass and Multi-surface Cleaners, Hard Water Mineral Removers, Metal Cleaners and Polishes, Oven Cleaners, Shower Cleaners—Daily, Toilet Bowl Cleaners, Tub, Tile and Sink Cleaners, room fresheners. Calcium polycarbophil, alginic acid component. It is in the scope of the invention wherein the concentration of the household agent is 0.0001% (wt/wt) or less. Alternatively, the range varies from about 0.0002% (wt/wt) to about 0.001% (wt/wt). Alternatively, the range varies from about 0.001% (wt/wt) to about 0.01% (wt/wt). Alternatively, the range varies from about 0.02% (wt/wt) to about 0.1% (wt/wt). Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

It is another embodiment of this invention wherein an agricultural agent is one or more members of a group consisting of Fungicide, Various prior cross-linked organic polymers used as a film former in the prior art for seed coating applications mainly include the cross-linked copolymer of acrylics, modified polyacrylamide and vinyl acrylic resins or the copolymers of polyvinyl acetate, methyl cellulose, etc. silicone polymers, ionic silicone. Pesticide: phenoxy acetic acids, phenoxy propionic acids, phenoxy butyric acids, benzoic acids, triazines and s-triazines, substituted ureas, uracils, bentazon, desmedipham, methazole, phenmedipham, pyridate, amitrole, clomazone, fluridone, norflurazone, dinitroanilines, isopropalin, oryzalin, pendimethalin, prodiamine, trifluralin, glyphosate, sulfonylureas, imidazolinones, clethodim, diclofop-methyl, fenoxaprop-ethyl, fluazifop-p-butyl, haloxyfop-methyl, quizalofop, sethoxydim, dichlobenil, isoxaben, and bipyridylium compounds. Fungicide compositions that can be used with the present invention include, but are not limited to, aldimorph, tridemorph, dodemorph, dimethomorph; flusilazol, azaconazole, cyproconazole, epoxiconazole, furconazole, propiconazole, tebuconazole and the like, imazalil, thiophanate, benomyl carbendazim, chlorothialonil, dicloran, trifloxystrobin, fluoxystrobin, dimoxystrobin, azoxystrobin, furcaranil, prochloraz, flusulfamide, famoxadone, captan, maneb, mancozeb, dodicin, dodine, and metalaxyl. Insecticide, larvacide, miticide and ovacide compounds that can be used with the composition of the present invention include, but are not limited to, Bacillus thuringiensis, spinosad, abamectin, doramectin, lepimectin, pyrethrins, carbaryl, primicarb, aldicarb, methomyl, amitraz, boric acid, chlordimeform, novaluron, bistrifluoron, triflumuron, diflubenzuron, imidacloprid, diazinon, acephate, endosulfan, kelevan, dimethoate, azinphos-ethyl, azinphos-methyl, izoxathion, chlorpyrifos, clofentezine, lambda-cyhalothrin, permethrin, bifenthrin, cypermethrin and the like. It is in the scope of the invention wherein the concentration of the agriculture agent is 0.0001% (wt/wt) or less. Alternatively, the range varies from about 0.0002% (wt/wt) to about 0.001% (wt/wt). Alternatively, the range varies from about 0.001% (wt/wt) to about 0.01% (wt/wt). Alternatively, the range varies from about 0.02% (wt/wt) to about 0.1% (wt/wt). Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

It is another embodiment of this invention wherein the wound dressing comprises a coating additive is one or more members of a group consisting of coating additive is selected from (b1) a water scavenger, (b2) a pigment, (b3) a diluent, (b4) a filler, (b5) a rust inhibitor, (b6) a plasticizer, (b7) a thickening agent, (b8) a pigment dispersant, (b9) a flow aid, (b10) a solvent, (b1 1) an adhesion promoter, (b12) a catalyst, (b13) an organic co-binder, (b14) a siloxane co-binder, (b15) a matting agent, (b16) a leveling agent, (b17) a wax, (b18) a texturizing additive, (b19) an anti-scratching additive, (b20) a gloss modifying additive, (b21) a stabilizer, and (b22) a croslinker, or a combination of two or more of (b1), (b2), (b3), (b4), (b5), (b6), (b7), (b8), (b9), (b10), (b11), (b12)(b13), (b14), (b15), (b16), (b17), (b18), (b19), (b20), (b21) and (b22). It is in the scope of the invention wherein the concentration of the coating agent is 0.0001% (wt/wt) or less. Alternatively, the range varies from about 0.0002% (wt/wt) to about 0.001% (wt/wt). Alternatively, the range varies from about 0.001% (wt/wt) to about 0.01% (wt/wt). Alternatively, the range varies from about 0.02% (wt/wt) to about 0.1% (wt/wt). Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

It is another embodiment of this invention wherein the wound dressing comprises stabilizers/preservatives being one or more members of a group consisting of, e.g., parahydroxybenzoic acid alkyl esters, antioxidants, antifungal agents, and the like; coloring agents, e.g., aluminum lake, titanium dioxide, and the like; excipients/disintegration modulating agents, e.g., magnesium silicate, silicic acid anhydride, aluminum silicate, calcium carbonate, magnesium aluminum metasilicate, calcium hydrogen phosphate, and the like; stearic acid and its salts; palmitic acid; talc; and other substances known as emulsifiers, dispersants, binders, thickeners and the like. It is in the scope of the invention wherein the concentration of the stabilizer is 0.0001% (wt/wt) or less. Alternatively, the range varies from about 0.0002% (wt/wt) to about 0.001% (wt/wt). Alternatively, the range varies from about 0.001% (wt/wt) to about 0.01% (wt/wt). Alternatively, the range varies from about 0.02% (wt/wt) to about 0.1% (wt/wt). Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

It is another embodiment of this invention wherein the wound dressing is utilized for the treatment of one or more medical or cosmetic indications elected from a group consisting of vaginal, anal, rectal, Injectable, proctology, dermal, buccal, growth factors, H acid, Trichology, NACS/NSID (anti-inflammatory), ontological preparation (EARS plug), anti-fungal (chlorohexidine), sun screens UV blockers, anti-aging serum, mucosal preparations, veterinary products.

It is another embodiment of this invention wherein the wound dressing comprises one or more members of a group consisting of spearmint oil, peppermint oil, cinnamaldehyde, cetyl pyridinium chloride, menthol saccharin sodium, glycyrrhizin, malt syrup, citric acid, tartaric acid, lemon oil, citrus flavor, and the like, sodium fluoride and the like, anti-plaque/anti-bacterial compositions suitable to treat or prevent periodontal disease, e.g., chlorobutanol, chlorothymol, chlorohexidine, their salts, and the like, dental pain control ingredients, e.g., benzocaine, lidocaine and the like; carbohydrates and hydrocolloids which act to modify the physical and chemical properties of the matrix. For example, an auxiliary hydrocolloid may be employed, such as cellulose polymers which are cellulose ethers such as methyl cellulose, cellulose alkyl hydroxylates such as hydroxypropylmethyl cellulose, hydroxypropyl cellulose, hydroxymethyl cellulose or hydroxyethyl cellulose, cellulose alkyl carboxylates such as carboxymethyl cellulose and carboxyethyl cellulose, and alkali metal salts of cellulose alkyl carboxylates, such as sodium carboxymethyl cellulose and sodium carboxyethyl cellulose, as well as carboxypolymethylene (molecular weight 2.5 to 3.5 million), gum acacia, guar gum, gum tragacanth, gum xanthan, alkali metal or alkaline earth metal carageenates, and alginates, such as alginic acid, ammonium or sodium alginate or mixtures thereof. Simple or complex carbohydrates or polyols, such as sucrose, xylose, mannitol, glucose, starch, Pluronic® surfactants, inorganic salts such as dicalcium phosphate, and the like, may also be employed to modify the hydrogel structure. It is in the scope of the invention wherein the concentration of the same is 0.0001% (wt/wt) or less. Alternatively, the range varies from about 0.0002% (wt/wt) to about 0.001% (wt/wt). Alternatively, the range varies from about 0.001% (wt/wt) to about 0.01% (wt/wt). Alternatively, the range varies from about 0.02% (wt/wt) to about 0.1% (wt/wt). Alternatively, the range varies from about 0.1% (wt/wt) to about 1.5% (wt/wt). Alternatively, the range varies from about 1.5% to about 5%. Alternatively, the range varies from about 5% to about 15%. Alternatively, the range varies from about 15% to about 35%. Alternatively, 35% or more.

1. A hydrogel wound dressing comprising one or more RTR components in a low viscosity aqueous solution and one or more non-RTR components.

2. The wound dressings of claim 1, wherein said one or more RTR components are configured to generate said wound dressing either or both (i) in situ, namely at time of deploying said composition onto a body region under conditions allowing formation of a solid or semi-solid dressing including the formation of a film; and (ii) a priori such a deployment of said composition onto a body region; said conditions are selected from a group consisting of amount of material, speed of deployment, method of deployment, exposure to radiation, including UV, gamma radiation and plasma treatment, e-beam emission, chemical reactions including cross linker admixture, exposure to temperature, oxygen, and any combination thereof.

3. The wound dressings of claim 1, wherein at least one RTR or layers or any other spatial arrangement thereof are interconnected between themselves and/or between one or more non-RTR components so as to form an integral part of said wound dressing.

4. The wound dressings of claim 1 , wherein at least one RTR component or layers or any other spatial arrangement thereof and at least one non-RTR component or layers thereof are not interconnected.

5. A biodegradable wound dressing of claim 1, wherein said biodegradability is tunable in time or in space.

6. The wound dressings of claim 1, wherein at least one first component is configured to respond to at least one first stimulus and at least one second component is configured to respond to at least one second stimulus, said stimulus is selected from a group consisting of temperature, time, water, oxygen concentration, NIR, IR, visible light or UV emission and any combination thereof.

7. The wound dressings of claim 1, wherein at least one component is configured to respond to one or more stimuli and release one or more components.

8. The wound dressings of claim 1, wherein at least one component comprises one or members of a group consisting of blends, semi-IPNs, IPNs, copolymers, derivatives, and any mixture or combination thereof.

9. The wound dressings of claim 1, further comprise at least one solid component that is configured to appear in a diversity of shapes, sizes and geometries, including one or more members of a group consisting of spheres, particles of any other shape, capsules, fibers, ribbons, films, meshes, fabrics, non-woven structures, foams, porous structures of different types, each of them having the possibility of being solid, porous, hollow and combinations thereof and having a size spanning from nanometric to centimetric.

10. The wound dressings of claim 1, further comprise a component of relevance, said relevancy is selected from a group consisting of pharmacological and/or biological relevance, including drugs and drug residues, oligopeptide sequences, growth factors, material containing genetic information and combinations thereof.

11. The wound dressings of claim 1, wherein said component is blended with any other component of the wound dressing, prior to, during or after deployment.

12. The wound dressings of claim 1, wherein polymeric chains displaying the temperature-dependent coiling-uncoiling ability in aqueous media are attached to the surface of a substrate, typically polymeric.

13. The wound dressings of claim 1, wherein RTR are polymers selected from a group consisting of one or more members of a group consisting of poly(ethylene oxide)-polypropylene oxide)-poly(ethylene oxide) (PEO-PPO-PEO) triblocks, random or alternating RTR PEO-PPO block copolymers, tetr afunctional block polymers of polyoxyethylene and polyoxypropylene condensed with ethylenediamine, N-alkyl substituted acrylamides (preferably poly-N-isopropyl acrylamide [PNIPAAm], cellulose derivatives, selected from a group consisting of hydroxypropyl methylcellulose and hydroxypropyl cellulose, alternating or random, and various amphiphilic polymers including poly(ethylene oxide)-polylactic acid block copolymers, and combination, mixture and derivative thereof.

14. The wound dressings of claim 1, wherein RTR chains are either or both surface grafted to the substrate or any other component of the dressing by reacting the RTR chains or their precursors to functional groups present on the substrate surface layer or on the surface of any of said other component/s of the dressing, or by generating reactive anchoring sites on the surface of any of them by various techniques selected from e-beam, UV or gamma radiation, chemical reactions and plasma treatments or mechanically stabilized on the surface layer of the substrate by physical interlocking generating a surface confined blend, semi-IPN or IPN.

15. The wound dressings of claim 1, wherein RTR polymers comprise either or both poly(ethylene oxide) and poly (propylene oxide) blocks.

16. A dressing comprising at least one first RTR and an active component (AC) integrated within said RTR.

17. The wound dressings of claim 16, wherein said AC is selected from at least one member of a group consisting of one or more INCI [i.e., cosmetics]; nutraceutical; medicament; cannabinoid, CBD, THC, cannabis and extracts thereof; household agent; agricultural agent; and industrial chemical agent.

18. A method of generating hydrogel wound dressings comprising providing one or more RTR components in a low viscosity aqueous solution and one or more non-RTR components.

19. The method of claim 18, comprising steps deploying said one or more RTR components thereby generating said wound dressing, either or both (i) in situ, namely at time of deploying said composition onto a body region under condition allowing formation of a solid or semi solid dressing including the formation of a film; and (ii) a priori said step of deploying of said composition onto a body region; said conditions are selected from a group consisting of amount of material, speed of deployment, method of deployment, exposure to radiation, including UV, gamma radiation and plasma treatment, e-beam emission, chemical reactions including cross linker admixture, exposure to temperature, oxygen, and any combination thereof.

20. The method of claim 18, comprising step of interconnecting at least one RTR or layers or any other spatial arrangement thereof between themselves and/or between one or more non-RTR components under condition allowing formation of a film and wound dressing; said condition is one or more member of a group consisting of admixing a cross-linker; applying irradiation of the type, duration and intensity enabling interconnection; exposing to oxygen and any combination thereof.

21-43. (canceled)
